(19) United States
(12) Patent Application Publication
(10) Pub. No.: US 2006/0286584 A1
(43) Pub. Date: Dec. 21, 2006

(54) Compositions and methods for the regulation of cell proliferation and apoptosis

(75) Inventor: Pan Duojia, Baltimore, MD (US)

Correspondence Address: CHALKER FLORES, LLP, 2711 LBJ FRWY, Suite 1036, DALLAS, TX, 75234, US

(73) Assignee: Board Of Regents, The University Of Texas System, Austin, TX (US)

(21) Appl. No.: 11/433,145

(22) Filed: May 12, 2006

Related U.S. Application Data
(60) Provisional application No. 60/680,997, filed on May 12, 2005.
(60) Provisional application No. 60/699,274, filed on Jul. 14, 2005.

(51) Int. Cl. C12Q 1/68; G01N 33/574; C07H 21/04; C12P 21/06; C12N 5/06; C40B 30/06; C07K 14/82; C07K 16/30

(52) U.S. Cl. 435/6; 536/23.5; 435/69.1; 435/320.1; 435/325; 530/350; 530/388.8; 435/7.23

(57) ABSTRACT

The present invention includes compositions and methods for the detection, characterization, diagnosis and kits related to the modification of transcriptional factors of the YAP signaling pathway and their correlation with oncogenesis.

### STATEMENT OF FEDERALLY FUNDED RESEACH

This invention was made with U.S. Government support under NIH grant ROI EY015708, the government may own certain rights in this invention.

### TECHNICAL FIELD OF THE INVENTION

The present invention relates in general to the field of compositions and methods for the regulation of cell proliferation and apoptosis, and more particularly, to methods, kits, nucleic acids and polypeptides for use in detecting and evaluating the status of cellular proliferation and apoptosis.

### BACKGROUND OF THE INVENTION

This application claims priority to U.S. Provisional Patent Applications Ser. No. 60/680,997, filed May 12, 2005 and Ser. No. 60/699,274 filed Jul. 14, 2005, the entire contents of which are incorporated herein by reference. Without limiting the scope of the invention, its background is described in connection with cell proliferation and apoptosis.

The increase in cell number that accompanies the growth of an organ or organism results from the balanced coordination of three simultaneous processes, including cell growth, cell proliferation and cell death (reviewed by Conlon and Raff, 1999; Hipfner and Cohen, 2004). Cell growth is a prerequisite for cell proliferation during normal organ growth and sustained cell proliferation must be coupled to appropriate cell growth. With appropriate cell growth, a net increase in cell number in a growing organ depends on the rate at which they are generated via cell proliferation, as well as the rate at which they are eliminated by cell death (apoptosis). How cell proliferation and cell death are coordinated during tissue growth and homeostasis is yet to be completely understood, and this mechanism must be intact throughout life to prevent diseases such as cancer.

Recent studies in mice and fruit flies have revealed two distinct modes in which cell proliferation and cell death could be coupled. In the first mode, increased proliferation, such as that resulting from activation of oncogenes Myc and Ras, is coupled in an obligatory fashion to increased cell death. Such coupling between proliferation and apoptosis provides an important failsafe mechanism to prevent inappropriate proliferation of somatic cells (reviewed by Lowe et al., 2004). In the second mode, increased proliferation, such as that resulting from activation of the microRNA bantam, or inactivation of the tumor suppressors hippo (hpo), Salvador (sav) and warts (wts), is accompanied by an inhibition of cell death (reviewed by Hipfner and Cohen, 2004; Hay and Guo, 2003; Ryoo and Steller, 2003). Here, suppression of cell death might allow the overproliferating cells to overcome proliferation-induced apoptosis, thus resulting in a robust increase in organ size. In many aspects, these circumstances resemble certain cancer cells, which display both increased cell proliferation and suppressed cell death (Lowe et al., 2004).

### SUMMARY OF THE INVENTION

The present invention relates to compositions and methods for the regulation of cell proliferation and apoptosis, and more particularly, to the regulation, characterization, isolation and development of agents that affect second messenger signaling through the Ste-20:YAP pathway.

The present invention includes a method for diagnosing changes in cellular proliferation including the steps of determining the level of phosphorylation of a yes-associated protein (YAP) of a sample cell; and comparing the level of phosphorylation of the yes-associated protein (YAP), e.g., yorkie (yki), in the sample cell to that of a normal cell, wherein a change in the level of phosphorylation is indicative of cell proliferation or cell death. As will be apparent to the skilled artisan, cellular proliferation is related to oncogenic potential, as such, by measuring the expression, longevity and/or post-translational modification of the proteins disclosed herein, it is possible to identify and treat certain cancers. For example, the yes-associated protein (YAP) is phosphorylated by a Wts/Lats protein kinase and may even be used to measure transcription of cyclin E and diap1 in an in vitro translation reaction. In one example, the phosphorylation may be measured in vitro. It has been found that yki overexpression leads to cell proliferation, as such, the present invention includes detection of not only the state of phosphorylation by also the level of expression of yki as ways to detect changes in cellular proliferation and/or cell morphology. For example, the sample cell may be obtained from a patient suspected of having cancer, e.g., a human patient.

The present invention also includes a method of identifying a polypeptide that interacts with yes-associated protein (YAP), by contacting a polypeptides from a cDNA expression library with a Wts protein; and identifying a polypeptide having a selective binding affinity for said recognition unit complex; wherein the binding specificity of the recognition units has been decreased by incorporating said recognition unit into said multivalent recognition unit complex.

Yet another embodiment of the present invention is a kit for detecting the extent of interaction between a yes-associated protein (YAP) and a Wts protein that includes one or more vials comprising the YAP and the Wts protein and a kit for detecting the level of phosphorylation of the YAP protein. The kit may be used along with a method for detecting an agent that interfere with the interaction between a yes-associated protein (YAP) and a Wts protein by contacting the YAP and the Wts protein with the agent and detecting the level of phosphorylation of the YAP protein, e.g., in vitro. The kit may also be used as a diagnostic tool for oncogenesis and/or oncogenic potential in samples that express the YAP nucleic acids and proteins disclosed herein. The YAP may be yorkie (yki), and YAP activity may be detected functionally using, e.g., in vitro translation of cyclin E, diap1 and combinations thereof. Alternatively, aberrant expression of YAP may be detected directly at the nucleic acid and/or protein level and correlated and/or diagnostic of oncogenesis and/or oncogenic potential. Another embodiment, includes a method of affecting cell growth and proliferation by delivering to a cell one or more expressible copies of a yes-associated protein in a vector.

The present invention also includes an isolated and purified nucleic acid that encodes the gene yki that is a yes-associated protein and may be oncogenic. Furthermore, an isolated and purified Yorkie polypeptide that is a yes-associated protein is also part of the present invention, which is related with oncogenesis. A method for detecting oncogenesis by measuring the level of expression of a yes-associated protein (YAP) from a cell, wherein changes in the expression of YAP is indicative of oncogenesis is also included. For example, the invention also includes an oncogene that is an isolated and purified yki gene, e.g., an isolated and purified nucleic acid that encodes an oncogene of SEQ ID NO.:1 or 2 or a protein derived therefrom, e.g., for use in the detection of increased expression or post-translational modification of the YAP protein, e.g., phoshorylation. When aberrantly expressed in cells, the YAP protein has now been linked to oncogenesis and oncogenic potential.

Coordination between cell proliferation and cell death is essential to maintain homeostasis in multicellular organisms. In Drosophila, these two processes are regulated by a pathway involving the Ste-20-like kinase Hippo (Hpo) and the NDR family kinase Warts (Wts; also called Lats). Hpo phosphorylates and activates Wts, which in turn, through unknown mechanisms, negatively regulates the transcription of cell cycle and cell death regulators such as cyclE and diap1. Here yorkie (yki), the Drosophila orthologue of the mammalian transcriptional co-activator yes-associated protein (YAP), is identified as a missing link between Wts and transcriptional regulation. Yki is required for normal tissue growth and diap1 transcription, and is phosphorylated and inactivated by Wts. Overexpression of yki phenocopies loss-of-function mutations of hpo or wts, including elevated transcription of cyclin E and diap1, increased proliferation, defective apoptosis and tissue overgrowth. Thus, Yki is a critical target of the Wts/Lats protein kinase and an oncogene.

Yet another embodiment of the present invention is an oncogene that is an isolated and purified yki gene of SEQ ID NO.:3. The oncogene may be, e.g., a wild-type yki or YAP gene that is overexpressed and/or that has been modified at the genotypic or phenotypic level and that triggers cellular proliferation. The isolated and purified nucleic acid may encodes an oncogene and have SEQ ID NO.:3. Another embodiment of the present invention includes a isolated and purified oncogenic protein of SEQ ID NO.:1 or 2. Other embodiments include a vector that includes may include a portion of the isolated and purified nucleic acid that encodes an oncogene of SEQ ID NO.:3, a specific probe that binds specifically to SEQ ID NO.:3 or the complement thereof and even a host cell with at least a portion or even the complete nucleic acid of SEQ ID NO.:3.

The present studies demonstrate that the biochemical activity of Yki and YAP are conserved, suggesting that like Yki, the YAP protein also critically controls organ size. Thus, like in Drosophila, one should be able to increase mammalian organ size and to decrease organ size by decreasing YAP activity. This will have applications in tissue and organ engineering. These observations may be used to demonstrate that YAP is one of the most potent oncogenes in mammals.

As such, the present invention also includes a method for modulating the size of an engineered organ by controlling the expressing of a nucleic acid of SEQ ID NO.: 3 to modify intracellular YAP activity. In some embodiments, the nucleic acid of SEQ ID NO.: 3 is overexpressed to increase YAP activity to increase organ size, while in alternative (but not mutually exclusive) embodiments the expression of the nucleic acid of SEQ ID NO.: 3 is decreased to reduce YAP activity to decrease organ size.

The present invention also includes a method for detecting oncogenicity by comparing the level of mRNA expression of a cell for SEQ ID NO.: 3 as compared to a wild-type cell, wherein an increase in the level of mRNA expression is indicative of oncogenesis. The oncogenic potential of the cell may be confirmed by detecting a decrease in YAP phosphorylation and/or decreased cellular apoptosis.

Many known oncogenes, including Ras and Myc, promote both cell proliferation and apoptosis (or senescence). Thus, the oncogenic potential of Ras or Myc is limited as compared to Yki and YAP, because Yki and YAP promote cell proliferation as well as inhibit apoptosis. Based on the present observation it is expected that many human cancers and tumors will show increased YAP activity, which can be reflected by either a higher expression level, or lower levels of phosphorylation, of the YAP protein. The present invention also includes compositions, methods and kits for the detection of cancer cells by detecting Yki and/or YAP, in situ, in vitro, after extraction and the like. Further, these studies show that YAP represents a new drug target for developing therapeutics against cancer.

A previous study reported that YAP is enriched in stem cells, although the functional significance of this enrichment is unclear (Ramalho-Santos et al., Science 298: 596-600 (2002)). The present studies suggest that the ability of YAP to promote cell proliferation and prohibit apoptosis might underlie the requirement of YAP in stem cells. Thus, YAP may be used to target stem cell-based therapies.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C summarize the steps in the identification of the yki gene. FIG. 1A shows the isolation of Yki as a Wts-binding protein by yeast two-hybrid screen. The cdc25H strain was transformed with the indicated plasmids and plated at permissive (25° C.) and restrictive (37° C.) temperature in the presence of galactose (prey expression induced) or glucose (prey expression suppressed). Sos-Wts is the bait plasmid used for library screening. Sos is empty bait vector containing only Sos protein. Myr is empty prey vector containing the myristylation signal only. 18-418, 186-418, 229-418 are three independent Yki clones isolated from the CytoTrap screen with the numbers representing the starting and ending position of the Yki polypeptides in the prey plasmids.

FIG. 1B demonstrates the physical association between Yki and Wts. HA-tagged wildtype Yki (Yki, lane 1), a mutant Yki carrying point mutations in the WW domains (Yki^{W292A P295A W361A P364A}, abbreviated as Yki^{WM} in lane 2), or a truncated Yki containing only the N-terminal half of the protein (Yki^{N}, lane 3) were co-expressed with V5-tagged Wts. α-HA immunoprecipitates were probed with and α-V5 antibody to detect physical interaction between the Yki variants and Wts. Note that wildtype Yki, but not Yki^{WM} or Yki^{N}, could immunoprecipitate Wts.

FIG. 1C is a sequence alignment of Yki with the human YAP protein. Yki is a 418 a.a. polypeptide, while YAP is a 504 a.a. polypeptide (SEQ ID NOS.: 1 and 2, respectively). The starting position of the three Yki clones identified in the yeast two-hybrid screen is indicated by arrows. WW domains are indicated by single underlines. The Pro-rich motif of YAP that binds to the SH3 domain of Yes (Sudol, 1994) is indicated by double underlines. The alanine mutations that were used to mutate the WW domains in Yki^{W292A P295A W361A P364A} are also indicated.

FIGS. 2A to 2J are figures that show that the overexpression of yki drives tissue overgrowth. In all panels, yki overexpression clones were generated using the Act>CD2>Gal4 flip-out technique coupled with UAS-GFP and UAS-yki transgenes.

FIG. 2A is a scanning electron micrograph (SEM) of Drosophila notum showing a large overgrowth (arrow) caused by a yki-overexpressing clone.

FIG. 2B is a high magnification view of epidermal cells near the border of a yki-overexpressing clone on the notum. The dashed line marks the border between the wildtype cells and the mutant clone. The mutant clone is located to the left of the border. Note the honeycomb-like appearance of the yki-overexpressing cells, which were not seen in the neighboring wildtype epidermal cells.

FIG. 2C shows that yki-overexpressing clones results in dramatic increase in the area of third instar wing discs. The inset at lower left corner shows a sibling control wing imaginal disc without yki-overexpression.

FIG. 2D is an image of a wing imaginal disc containing 48 hr-old control clones generated by flip-out and positively marked by GFP. Note the irregular, wavy outline of the marked clones.

FIG. 2E is an image of a wing imaginal disc containing 48 hr-old yki-overexpressing clones generated by flip-out and positively marked by GFP. yki-overexpressing clones grow larger than control clones (compare 2E and 2D). Also note the round outline of the yki-overexpressing clones.

FIG. 2F shows the wing imaginal disc containing 48 hr-old control clones generated by FLP/FRT and marked by the absence of GFP. Note the irregular, wavy outline of the marked clones.

FIG. 2G shows a wing imaginal disc containing 48 hr-old wts mutant clones generated by FLP/FRT and marked by the absence of GFP. Note the larger size and the round circumference of the wts mutant clones.

FIG. 2H is a graph of a flow cytometric analysis of dissociated wing imaginal discs containing yki-overexpressing clones. The DNA profiles of wildtype and mutant cells are indicated by red and green traces, respectively. The inset shows forward scattering (FSC), which measures cell size.

FIGS. 2I-I″ are third instar eye disc containing a yki-overexpressing clone (marked positively by GFP) and stained for the neuronal specific Elav protein (red). Three images are shown, one of GFP (FIG. 2I), one of Elav (FIG. 2I′) and one of superimposed GFP and Elav (FIG. 2I″). Note the presence of Elav-positive photoreceptor clusters in yki-overexpressing clone (indicated by arrows), as well as increased spacing between photoreceptor clusters in the clone. Arrowhead marks the MF.

FIGS. 2J-J′″ show a 40 hr pupal eye with a yki-overexpressing clone (marked positively by GFP) and stained for phalloidin (red), which highlights the outlines of the cells, and Armadillo (blue), which at this focal plane labels the apical cell surface of photoreceptors. Four images are shown, one of GFP (FIG. 2J), one of phalloidin (FIG. 2J′), one of Armadillo (FIG. 2J″) and one of superimposed GFP, phalloidin and Armadillo (FIG. 2J″′). Note the presence of a normal complement of photoreceptors and supernumerary interommatidial cells in the yki-overexpressing clone (indicated by arrows), as well as the increased spacing between photoreceptor clusters.

FIGS. 3A to 3F show that the overexpression of yki promotes cell proliferation and inhibits apoptosis. In all panels, yki-overexpression clones were generated using the Act>CD2>Gal4 flip-out technique in conjunction with UAS-GFP and UAS-yki transgene. yki-overexpression clones are positively marked by GFP and indicated by arrows.

FIG. 3A shows a wildtype third instar eye disc labeled by BrdU incorporation (red). Note that the S-phase cells are normally detected in a single band of cells in the SMW (arrowhead).

FIGS. 3B-B″ show third instar eye disc containing yki-overexpressing clones (marked positively by GFP) and labeled with BrdU incorporation (red). Three images are shown, one of GFP (3B), one of BrdU (3B′) and one of superimposed GFP and BrdU (3B″). Note that BrdU incorporation continues posterior to SMW in yki-overexpressing clones. Arrowhead marks the SMW.

FIGS. 3C-C″ show a 36 hr APF pupal eye containing yki-overexpressing clones and labeled by TUNEL staining. Three images are shown, one of GFP (green, B), one of BrdU (red, B′) and one of superimposed GFP and BrdU (B″). Cell death is absent in yki-overexpressing clones but abundant in the neighboring wildtype cells.

FIGS. 3D-D″ show a third instar eye disc containing yki-overexpressing clones (green) and stained with α-DIAP1 antibody (red). Note the cell autonomous increase in DIAP1 protein levels in yki-overexpressing cells.

FIGS. 3E-E″ show that yki-overexpressing clones (green) were generated in flies containing the diap1-lacZ reporter th^{j5c8}, and stained for lacZ protein (red). Note the cell-autonomous increase in diap1-lacZ expression in yki-overexpressing cells.

FIG. 3F-F″ show that yki-overexpressing clones (green) were generated in flies containing a cycE-lacZ reporter, and stained for lacZ protein (red). Arrowhead marks the MF. Note a modest increase in cycE-lacZ expression in yki-overexpressing cells (arrows).

FIGS. 4A to 4F show that yki is required for tissue growth and transcriptional regulation of diap1. FIG. 4A shows the generation of yki knockout allele by homologous recombination. The diagram at left shows genomic organization of the endogenous yki locus, the targeting construct and the expected targeted allele. DNA probe used for Southern blotting is shown as a solid line, and the sizes (in kilobases) of expected BamHI fragments are also indicated. The right shows Southern blotting of BamHI-digested genomic DNA hybridized with the indicated probe. DNA was extracted from wildtype flies (w¹¹¹⁸) and three independent yki knockout lines recovered from homologous recombination (as heterozygotes over the CyO balancer). The size of molecular weight marker is indicated to the right of the autoradiograph. While the endogenous yki locus produces a 10.7 kb fragment, the targeted allele produces 12.7 kb band. All the knockout alleles were completely rescued by a yki rescue construct.

FIGS. 4B-B′ are scanning electron micrographs (SEM) of wildtype flies showing a dorsal view of the head (FIG. 4B) and a side view of the compound eye (FIG. 4B′). The genotype is: y w ey-flp; FRT42D/FRT42D w⁺l(2)c1-R11

FIGS. 4C-C′ are SEMs of fly heads composed predominantly of cells lacking yki function. A dorsal view (FIG. 4C) and a side view (FIG. 4C′) are shown. The genotype is: y w ey-flp; FRT42D yki^{B5}/FRT42D w⁺l(2)c1-R11.

FIG. 4D-D″ shows a third instar wing disc containing yki mutant clones and stained with propidium iodide (PI). Mutant clones were induced by FLP/FRT at 40 hr AED and analyzed at 120 hr AED. Three images are shown, one of GFP (FIG. 4D), one of the nuclear stain propidium iodide (PI, FIG. 4D′) and one of superimposed GFP and propidium iodide (FIG. 4D″). Homozygous −/− clones, marked by the absence of GFP, were largely undetectable, with rare exceptions containing 1-3 cells (white arrow). In contrast, the sibling +/+ twin spots, marked by the 2XGFP signal (darker green, yellow arrow), were readily observed.

FIGS. 4E-E″ show the third instar eye disc with yki mutant clones (marked by lack of GFP) and stained for the neuronal specific Elav protein (red). Mutant clones were induced at 60 hr AED to allow for more efficient recovery of yki mutant cells. Three images are shown, one of GFP (FIG. 4E), one of Elav (FIG. 4E′) and one of superimposed GFP and Elav (FIG. 4E″). Note the presence of Elav-positive cells in yki mutant clones (arrow). Arrowhead marks the MF.

FIGS. 4F-F′″ show the effect of yki mutant clones (marked by lack of GFP) generated in flies containing the diap1-lacZ reporter th^{j5c8}, and a third instar eye disc was stained for lacZ (red) and the nuclear stain PI (blue). Four images are shown, one of GFP (FIG. 4F), one of diap1-lacZ (FIG. 4F′), one of PI (FIG. 4F″) and one of superimposed GFP, diap1-lacZ and PI (FIG. 4F″′). Note the cell autonomous decrease in diap1-lacZ levels in yki mutant clones (white arrow), and the smaller size of the yki mutant clone as compared to the twin spots (yellow arrow). Arrowhead marks the MF.

FIGS. 5A to 5D show that the Hpo signaling pathway antagonizes the transcriptional co-activator activity of Yki.

FIG. 5A is a schematic diagram of Gal4-related constructs used in the S2 cell transcription assay.

FIG. 5B is a graph that shows the co-activator activity of Yki and its negative regulation by the Hpo-Wts pathway. S2 cells were transfected with the indicated plasmids along with a Gal4-responsive luciferase reporter. The luciferase activities relative to those obtained with Gal4 DNA binding domain alone are plotted.

FIG. 5C is a graph that shows the antagonistic, dosage-sensitive, genetic interactions between yki and hpo-wts. The percentage of flies surviving to adults is shown for the indicated genotypes (>200 animals scored for each genotype). Note the complete lethality of GMR-hpo; GMR-wts animals and the complete reversal of lethality by GMR-yki. Also note a complete reversal of lethality by expression of the human YAP gene under the GMR-Gal4 driver (GMR-Gal4; UAS-YAP).

FIG. 5D is a graph that shows the antagonistic, dosage-sensitive, genetic interactions between yki and hpo. The percentage of flies surviving to adults is shown for the indicated genotypes (>200 animals scored for each genotype). The lethality of GMR-GAL4; UAS-hpo animals was completely and partially rescued by yki and YAP, respectively.

FIG. 6A to 6E show the phosphorylation of Yki by Wts.

FIG. 6A is a lysates from S2 cells expressing various epitope-tagged proteins were probed with indicated antibodies. Transfection of Wts or Hpo-Sav results in mobility shift of the co-expressed Yki protein (lanes 1-3). Also note the supershift of Yki when all three tumor suppressors are co-expressed (lane 4). Increasingly phosphorylated forms of Yki are indicated by small circles next to the protein bands, and filled with white, grey and black colors respectively.

FIG. 6B shows that phosphatase (CIP) treatment reversed the mobility shift of Yki induced by Hpo-Sav-Wts.

FIG. 6C shows that Wts phosphorylates Yki in vitro. V5-tagged Wts or kinase-dead Wts^{K743R} was expressed alone or together with Hpo-Sav in S2 cells, immunoprecipitated, and tested for kinase activity against GST-Yki and GST-Tsc1 (as a control substrate). The signal of Yki phosphorylation by Wts is indicated by arrowheads, and the arrow marks the expected migration position of the GST-Tsc1 on autoradiograph (top gel). The input kinase and substrate are also shown (bottom two gels). Note that the GST-Yki preparation contains two Yki-related bands (arrowheads) as well as a contaminating band (black dots, see for details). Note that only the Yki-related bands are phosphorylated, and that Yki phosphorylation is only detected when Wts is co-expressed with Hpo-Sav (lane 2).

FIG. 6D shows that the mobility shift of Yki induced by Hpo-Sav requires the endogenous Wts activity in S2 cells. S2 cells were transfected with the indicated plasmids along with control or wts dsRNA. Cell lysates were probed with indicated antibodies. Note suppression of Yki mobility shift by wts RNAi.

FIG. 6E shows that the mobility shift of Yki induced by Wts requires the endogenous Hpo activity in S2 cells. S2 cells were transfected with the indicated plasmids along with control or hpo dsRNA. Cell lysates were probed with indicated antibodies. Note suppression of Yki mobility shift by hpo RNAi.

FIGS. 7A to 7G show that yki is genetically epistatic to hpo, sav and wts. In all panels, the respective mutant clones were generated at 40 hr AED and marked by the lack of GFP. A-D″ show eye imaginal discs, and E-G show wing imaginal discs.

FIGS. 7A-A″ show the third instar eye disc with yki mutant clones in the presence the diap1-lacZ reporter th^{j5c8}, and with antibodies against DIAP1 (blue) and lacZ (red). Three images are shown, one of GFP (FIG. 7A), one of DIAP1 (FIG. 7A′) and one of lacZ (FIG. 7A″). Note the cell autonomous decrease of DIAP1 and diap1-lacZ levels in yki mutant clones (arrows). Also note the growth disadvantage of yki mutant clones as compared to the twin spots (marked by 2XGFP signal).

FIGS. 7B-B″, FIGS. 7C-C″ and FIGS. 7D-D″) are similar to (FIG. 7A-A″) except that double mutant clones of hpo yki (FIG. 7B-B″), sav yki (FIG. 7C-C″) and wts yki (FIG. 7D-D″) were analyzed in the eye imaginal discs. Note the cell autonomous decrease of DIAP1 and diap1-lacZ levels in these double mutant clones (representative clones indicated by arrows). Also note the growth disadvantage of the mutant clones as compared to the twin spots (marked by 2XGFP signal). These phenotypes resemble yki mutant clones (see FIGS. 4F-F″, FIG. 7A-A″).

FIGS. 7E and 7G show the third instar wing discs from double mutant clones of hpo yki (7E), sav yki (FIG. 7F) and wts yki (FIG. 7G). Note the presence of +/+ twin spots, marked by 2XGFP signal (darker green, yellow arrow), and a general absence of homozygous mutant cells (marked by the absence of GFP). This phenotype is similar to yki wing clones generated at 40 hr AED (see FIGS. 4D-D″).

FIG. 8 is a model of the Hpo signaling pathway in the control of organ growth. An unknown signal (marked by question mark) activates Hpo, which in turn phosphorylates and activates Wts. The activation of Wts by Hpo is potentiated by Sav and Mats, which have been shown to associate with Hpo and Wts, respectively. The activated Wts kinase phosphorylates and inactivates the transcriptional co-activator Yki, which normally partners with a downstream DNA binding transcription factor (X) to activate gene transcription. Transcriptional targets of the Yki-X transcription complex include the cell death gene diap1 and possibly cell cycle regulators such as cyclin E. In addition, other transcriptional target(s) of the Yki-X complex will regulate cell growth, since cell growth must be proportionally stimulated to sustain the increased proliferation of hpo, sav, wts and mats mutant cells.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used throughout the present specification the following abbreviations are used: TF, transcription factor; ORF, open reading frame; kb, kilobase (pairs); UTR, untranslated region; kD, kilodalton; PCR, polymerase chain reaction; RT, reverse transcriptase.

As used herein “a yes-associated protein-specific nucleic acid” or YAP is DNA, RNA, single-stranded or double-stranded and any chemical modifications thereof or protein nucleic acids that encode a YAP protein. Modifications include, but are not limited to, those that provide other chemical groups that incorporate additional charge, polarizability, hydrogen bonding and/or electrostatic interactions to individual nucleic acid bases or to nucleic acids as a whole.

A “nucleic acid target element” is a determinable sequence that contains at least one peptide located at a different location on the substrate. The determinable sequence comprises either DNA, RNA, single-stranded or double-stranded and any chemical modifications thereof. Modifications include, but are not limited to, those that provide other chemical groups that incorporate additional charge, polarizability, hydrogen bonding, electrostatic interaction, and fluxionality to the individual nucleic acid bases or to the nucleic acid as a whole. The determinable sequence can further be portions of structural, metabolic, transcriptional or other genes, including ones that code for a proteases, receptors, channels, synaptic proteins, cell-cell or cell-matrix interactions, immune or inflammatory responses, cell signaling, molecular chaperones or other carrier proteins, molecular synthesis, cell cycle regulation, cell growth, cell proliferation, or cell death.

A sample is any mixture of macromolecules obtained from a person. This includes, but is not limited to, blood, plasma, urine, semen, saliva, lymph fluid, meningeal fluid, amniotic fluid, glandular fluid, and cerebrospinal fluid. This also includes experimentally separated fractions of all of the preceding. “Sample” also includes solutions or mixtures containing homogenized solid material, such as feces, cells, tissues, and biopsy samples. Samples herein include one or more that are obtained at any point in time, including diagnosis, prognosis, and periodic monitoring.

The terms “a sequence essentially as set forth in SEQ ID NO. (#)”, “a sequence similar to”, “nucleotide sequence” and similar terms, with respect to nucleotides, refers to sequences that substantially correspond to any portion of the sequence identified herein as SEQ ID NO.: 3. These terms refer to synthetic as well as naturally-derived molecules and includes sequences that possess biologically, immunologically, experimentally, or otherwise functionally equivalent activity, for instance with respect to hybridization by nucleic acid segments, or the ability to encode all or portions of yki and having oncogenic activity, that modify YES-protein phosphorylation or a yes-associated protein (YAP) and/or a combination thereof. Naturally, these terms are meant to include information in such a sequence as specified by its linear order.

As used herein, the term “homology” refers to the extent to which two nucleic acids are complementary. There may be partial or complete homology. A partially complementary sequence is one that at least partially inhibits a completely complementary sequence from hybridizing to a target nucleic acid and is referred to using the functional term “substantially homologous.” The degree or extent of hybridization may be examined using a hybridization or other assay (such as a competitive PCR assay) and is meant, as will be known to those of skill in the art, to include specific interaction even at low stringency.

An oligonucleotide sequence that is “substantially homologous” to the oncogene of SEQ ID NO.: 3“is defined herein as an oligonucleotide sequence that exhibits greater than or equal to 75% identity to the sequence of SEQ ID NO.: 3 when sequences having a length of 100 bp or larger are compared and is referred to herein as the YAP protein.

As used herein, the term “gene” refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “vector” is used in reference to nucleic acid molecules that transfer DNA segment(s) from one cell to another. The vector may be further defined as one designed to propagate specific sequences, or as an expression vector that includes a promoter operatively linked to the specific sequence, or one designed to cause such a promoter to be introduced. The vector may exist in a state independent of the host cell chromosome, or may be integrated into the host cell chromosome

As used herein, the term “host cell” refers to cells that have been engineered to contain nucleic acid segments or altered segments, whether archeal, prokaryotic, or eukaryotic. Thus, engineered, or recombinant cells, are distinguishable from naturally occurring cells that do not contain recombinantly introduced genes through the hand of man.

As used herein, the term “agonist” refers to a molecule that enhances either the strength or the time of an effect of an oncogene that includes the amino acid sequences of SEQ ID NO.: 1 or 2. The term “antagonist” refers to a molecule that decreases either the strength or the time of an effect of oncogenic polypeptide and/or a yes-associated protein that encompasses small molecules, proteins, nucleic, acids, carbohydrates, lipids, or other compounds and that affect the activity Yes-associated proteins.

As used herein, the terms “altered”, or “alterations” or “modified” with reference to nucleic acid or polypeptide sequences are meant to include changes such as insertions, deletions, substitutions, fusions with related or unrelated sequences, such as might occur by the hand of man, or those that may occur naturally such as polymorphisms, alleles and other structural types. Alterations encompass genomic DNA and RNA sequences that may differ with respect to their hybridization properties using a given hybridization probe. Alterations of polynucleotide sequences for SEQ ID NO. 3, or fragments thereof, include those that increase, decrease, or have no effect on functionality. Alterations of polypeptides refer to those that have been changed by recombinant DNA engineering, chemical, or biochemical modifications, such as amino acid derivatives or conjugates, or post-translational modifications.

As used herein, the term “control sequences” refers to DNA sequences necessary for the expression of an operably linked coding sequence in a particular host organism. The control sequences that are suitable for prokaryotes, for example, include a promoter, optionally an operator sequence, a ribosome binding site, and transcriptional terminators. Highly regulated inducible promoters that suppress Fab′ polypeptide synthesis at levels below growth-inhibitory amounts while the cell culture is growing and maturing, for example, during the log phase may be used.

As used herein, when a nucleic acid is “operably linked” it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it effects the transcription of the sequence; or a ribosome binding site is operably linked to e coding sequence if it is positioned so as to facilitate translation. Generally, “operably linked” means that the DNA sequences being linked are contiguous and, in the case of a secretory leader, contiguous and in same reading frame. Enhancers do not have to be contiguous. Linking is accomplished by ligation at convenient restriction sites. If such sites do not exist, then synthetic oligonucleotide adaptors or linkers are used in accord with conventional practice.

As used herein, the term “exogenous” refers to a nucleic acid sequence that is foreign to the cell, or homologous to the cell but in a position within the host cell nucleic acid in which the element is ordinarily not found.

As used herein, the expressions “cell” and “cell culture” are used interchangeably to describe one or more cells and their progeny that may live, be grown and/or replicate in culture. The words “transformants” and “transformed cells” include a primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Different designations are will be clear from the contextually clear.

As used herein, the term “plasmids” refers to independently replicating extrachromosomal nucleic acid segments and are often referred to by a lower case “p” preceded and/or followed by capital letters and/or numbers. The starting plasmids herein are commercially available, are publicly available on an unrestricted basis, or can be constructed from such available plasmids in accord with published procedures. In addition, other equivalent plasmids are known in the art and will be apparent to the ordinary artisan.

As used herein, the term “aptamer” refers to an oligonucleotide that has been designed or discovered that is able to specifically bind a target sequence. The term aptazyme is used to describe an aptamer that also contains catalytic activity against nucleic acids or other targets.

As used herein, the term “protein-protein complex” or “protein complex” refers to an association of more than one protein. The proteins of the complex may be associated by a variety of means, or by any combination of means, including but not limited to functional, stereochemical, conformational, biochemical, or electrostatic association. It is intended that the term encompass associations of any number of proteins.

As used herein the terms “protein”, “polypeptide” or “peptide” refer to compounds comprising amino acids joined via peptide bonds and are used interchangeably.

As used herein, the term “endogenous” refers to a substance the source of which is from within a cell. Endogenous substances are produced by the metabolic activity of a cell. Endogenous substances, however, may nevertheless be produced as a result of manipulation of cellular metabolism to, for example, make the cell express the gene encoding the substance.

As used herein, the term “exogenous” refers to a substance the source of which is external to a cell. An exogenous substance may nevertheless be internalized by a cell by any one of a variety of metabolic or induced means known to those skilled in the art.

As used herein, the term “gene” refers to a functional protein, polypeptide or peptide-encoding unit. As will be understood by those in the art, this functional term includes genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “sequences” as used herein is used to refer to nucleotides or amino acids, whether natural or artificial, e.g., modified nucleic acids or amino acids. When describing “transcribed nucleic acids” those sequence regions located adjacent to the coding region on both the 5′, and 3′, ends such that the deoxyribonucleotide sequence corresponds to the length of the full-length mRNA for the protein as included. The term “gene” encompasses both cDNA and genomic forms of a gene. A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA I wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed, excised or “spliced out” from the nuclear or primary transcript; introns therefore are absent in the messenger RNA (mRNA) transcript. The mRNA functions during translation to specify the sequence or order of amino acids in a nascent polypeptide. In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation. DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand.

As used herein, the term “gene of interest” refers to a gene, the function and/or expression of which is desired to be investigated, or the expression of which is desired to be regulated, by the present invention. In the present disclosure, the xx gene of the xx is an example of a gene of interest and is described herein to illustrate the invention. The present invention may be useful in regard to any gene of any organism, whether of a prokaryotic or eukaryotic organism.

As used herein, the term “hybridize” refers to any process by which a strand of nucleic acid binds with a complementary strand through base pairing. Hybridization and the strength of hybridization (i.e., the strength of the association between the nucleic acid strands) is impacted by such factors as the degree of complementary between the nucleic acids, stringency of the conditions involved, the melting temperature of the formed hybrid, and the G:C (or U:C for RNA) ratio within the nucleic acids.

As used herein, the terms “complementary” or “complementarity” refer to the natural binding of polynucleotides under permissive salt and temperature conditions by base-pairing. For example, for the sequence “A-G-T” binds to the complementary sequence “T-C-A”. Complementarity between two single-stranded molecules may be partial, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands.

As used herein, the term “homology,” refers to a degree of complementarity. There may be partial homology or complete homology (i.e., identity). A partially complementary sequence is one that at least partially inhibits an identical sequence from hybridizing to a target nucleic acid; it is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence.

As used herein, the term “knock-outs,” e.g., conditional knock-outs, refers to the alteration of a target gene can be activated by exposure of the animal to a substance that promotes target gene alteration, introduction of an enzyme that promotes recombination at the target gene site (e.g., Cre in the Cre-lox system), or other method for directing the target gene alteration.

As used herein, the term “knock-in” refers to a target gene is used herein to define an alteration in a host cell genome that results in altered expression (e.g., increased or decreased expression) of a target gene, e.g., by introduction of an additional copy of the target gene, or by operatively inserting a regulatory sequence that provides for enhanced expression of an endogenous copy of the target gene. “Knock-in” transgenics include heterozygous knock-in of the target gene or a homozygous knock-in of a target gene and include conditional knock-ins.

When used in reference to a nucleic acid sequence, the term “substantially homologous” refers to any nucleic acid sequence or probe that hybridizes (i.e., it is the complement of) to a single-stranded nucleic acid sequence under conditions of low, medium or high stringency as is well-known to the skilled artisan. As known in the art, numerous equivalent conditions may be employed to comprise either low or high stringency conditions. Factors that affect binding specificity include the length and nature (DNA, RNA, base composition) of the sequence, nature of the target (DNA, RNA, base composition, presence in solution or immobilization, etc.), and the concentration of the salts and other components (e.g, the presence or absence of formamide, dextran sulfate and/or polyethylene glycol) are considered and the hybridization solution may be varied to generate conditions of either low or high stringency different from, but equivalent to, the above listed conditions.

As used herein, the term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less. Low stringency conditions comprise conditions equivalent to binding or hybridization at 42° C. in a solution consisting of 5× SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharmacia), 5 g BSA [Fraction V; Sigma]) and 100 μg/ml denatured salmon sperm DNA) followed by washing in a solution comprising 5× SSPE, 01% SDS at 42° C. when a probe of about 500 nucleotides in length is employed. High stringency conditions comprise conditions equivalent to binding or hybridization at 65° C. in a solution consisting of 5× SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharmacia), 5 g BSA [Fraction V; Sigma]) and 100 μg/ml denatured salmon sperm DNA) followed by washing in a solution comprising 5× SSPE, 01% SDS at 65° C. when a probe of about 500 nucleotides in length is employed. Numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions which promote hybridization under conditions of high stringency (e.g., increasing the temperature of the hybridization and/or wash steps, the use of formamide in the hybridization solution, etc.).

As used herein, the term “antisense,” refers to nucleotide sequences that are complementary to a specific DNA or RNA sequence. The term “antisense strand” is used in reference to a nucleic acid strand that is complementary to tile “sense” strand. Antisense molecules may be produced by any method, including synthesis by ligating the gene(s) of interest in a reverse orientation to a viral promoter which permits the- synthesis of a complementary strand. Once introduced into a cell, the transcribed strand combines with natural sequences produced by the cell to form duplexes. These duplexes then block either the further transcription or translation. In this manner, mutant phenotypes may also be generated. The designation “negative” is sometimes used in reference to the antisense strand, and “positive” is sometimes used in reference to the sense strand. The term also is used in reference to RNA sequences that are complementary to a specific RNA sequence (e.g., mRNA). Included within this definition are antisense RNA (“asRNA”) molecules involved in genetic regulation by bacteria. Antisense RNA may be produced by any method, including synthesis by splicing the gene(s) of interest in a reverse orientation to a viral promoter which permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand with the designation (+) sometimes used in reference to the sense (i.e., “positive”) strand.

As used herein, the term “transformation,” refers to a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome.

As used herein, the term “transfection” refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell which has stably integrated foreign DNA into the genomic DNA. The term also encompasses cells which transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA.

As used herein, the term “selectable marker” refers to the use of a gene that encodes an enzymatic activity and which confers the ability to grow in medium lacking what would otherwise be an essential nutrient (e.g., the HIS3 gene in yeast cells); in addition, a selectable marker may confer resistance to an antibiotic or drug upon the cell in which the selectable marker is expressed. A review of the use of selectable markers in mammalian cell lines is provided in Sambrook, J., et al., MOLECULAR CLONING: A LABORATORY MANUAL, 2nd ed., Cold Spring Harbor Laboratory Press, New York (1989) pp.16.9-16.15.

As used herein, the term “reporter gene” refers to a gene that is expressed in a cell upon satisfaction of one or more contingencies and which, upon expression, confers a detectable phenotype to the cell to indicate that the contingencies for expression have been satisfied. For example, the gene for Luciferase confers a luminescent phenotype to a cell when the gene is expressed inside the cell. In the present invention, the gene for Luciferase may be used as a reporter gene such that the gene is only expressed upon the splicing out of an intron in response to an effector. Those cells in which the effector activates splicing of the intron will express Luciferase and will glow.

As used herein, the term “vector” refers to nucleic acid molecules that transfer DNA segment(s) from one cell to another. The term “vehicle” is sometimes used interchangeably with “vector.” The term “vector” as used herein also includes expression vectors in reference to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “amplify”, when used in reference to nucleic acids, refers to the production of a large number of copies of a nucleic acid sequence by any method known in the art. Amplification is a special case of nucleic acid replication involving template specificity. Template specificity is frequently described in terms of “target” specificity. Target sequences are “targets” in the sense that they are sought to be sorted out from other nucleic acid. Amplification techniques have been designed primarily for this sorting out.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer may be single stranded for maximum efficiency in amplification but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target” when used in reference to the polymerase chain reaction, refers to the region of nucleic acid bounded by the primers used for polymerase chain reaction. Thus, the “target” is sought to be sorted oat from other nucleic acid sequences. A “segment” is defined as a region of nucleic acid within the target sequence.

As used herein, the term “polymerase chain reaction” (“PCR”) refers to the methods taught in, e.g., U.S. Pat. Nos. 4,683,195, 4,683,202, and 4,965,188, relevant portions incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

The word “specific” as commonly used in the art has two somewhat different meanings. The practice is followed herein. “Specific” refers generally to the origin of a nucleic acid sequence or to the pattern with which it will hybridize to a genome, e.g., as part of a staining reagent. For example, isolation and cloning of DNA from a specified chromosome results in a “chromosome-specific library”. Shared sequences are not chromosome-specific to the chromosome from which they were derived in their hybridization properties since they will bind to more than the chromosome of origin. A sequence is “locus specific” if it binds only to the desired portion of a genome. Such sequences include single-copy sequences contained in the target or repetitive sequences, in which the copies are contained predominantly in the selected, sequence.

As used herein, the term “staining reagent” refers to the overall hybridization pattern of the nucleic acid sequences that comprise the reagent. A staining reagent that is specific for a portion of a genome provides a contrast between the target and non-target chromosomal material. A number of different aberrations may be detected with any desired staining pattern on the portions of the genome detected with one or more colors (a multi-color staining pattern) and/or other indicator methods.

As used herein, the term “labeled” refers to an agent or agents that permit a user to visualize or detect a target using a probe, whether or not the probe directly carries some modified constituent. The terms “staining” or “painting” are herein defined to mean hybridizing a probe of this invention to a genome or segment thereof, such that the probe reliably binds to the targeted region or sequence of chromosomal material and the bound probe is capable of being detected. The terms “staining” or “painting” are used interchangeably. The patterns on the array resulting from “staining” or “painting” are useful for cytogenetic analysis, more particularly, molecular cytogenetic analysis. The staining patterns facilitate the high-throughput identification of normal and abnormal chromosomes and the characterization of the genetic nature of particular abnormalities.

Multiple methods of probe detection may be used with the present invention, e.g., the binding patterns of different components of the probe may be distinguished--for example, by color or differences in wavelength emitted from a labeled probe.

As used herein, the term “transgene” refers to genetic material that may be artificially inserted into a mammalian genome, e.g., a mammalian cell of a living animal. The term “transgenic animal is used herein to describe a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal according to methods well known in the art.

As used herein, the term “transgene” refers to such heterologous nucleic acid, e.g., heterologous nucleic acid in the form of, e.g., an expression construct (e.g., for the production of a “knock-in” transgenic animal) or a heterologous nucleic acid that upon insertion within or adjacent a target gene results in a decrease in target gene expression (e.g., for production of a “knock-out” transgenic animal). A “knock-out” of a gene means an alteration in the sequence of the gene that results in a decrease of function of the target gene, preferably such that target gene expression is undetectable or insignificant. Transgenic knock-out animals include a heterozygous knock-out of a target gene, or a homozygous knock-out of a target gene.

As used herein, the term “stem cell” refers to pluripotent stem cells, e.g., embryonic stem cells, and to such pluripotent cells in the very early stages of embryonic development, including but not limited to cells in the blastocyst stage of development.

As used herein, the term “wild type” sequence refers to a coding, non-coding or interface sequence is an allelic form of sequence that performs the natural or normal function for that sequence. Wild type sequences include multiple allelic forms of a cognate sequence, for example, multiple alleles of a wild type sequence may encode silent or conservative changes to the protein sequence that a coding sequence encodes. As used herein, the term “mutant” sequence refers to one in which at least a portion of the functionality of the sequence has been lost, for example, changes to the sequence in a promoter or enhancer region will affect at least partially the expression of a coding sequence in an organism. As used herein, the term “mutation,” refers to any change in a sequence in a nucleic acid sequence that may arise such as from a deletion, addition, substitution, or rearrangement. The mutation may also affect one or more steps that the sequence is involved in. For example, a change in a DNA sequence may lead to the synthesis of an altered mRNA and/or a protein that is active, partially active or inactive.

The present invention includes the demonstration that the biochemical activity of Yki and YAP are conserved, suggesting that like Yki, the YAP protein controls critically organ size. Thus, like in Drosophila, one should be able to increase mammalian organ size by increasing YAP activity and to decrease organ size by decreasing YAP activity. This will have applications in tissue and organ engineering. These studies also demonstrate that YAP is one of the most potent oncogenes in mammals.

Many known oncogenes, including Ras and Myc, promotes both cell proliferation and apoptosis (or senescence). Thus, their oncogenic potentials are limited, as compared to Yki and YAP, which promote cell proliferation as well as inhibit apoptosis. Based on these activities human cancers and tumors are expected to show increased YAP activity, which can be reflected by either a higher expression level, or lower phosphorylation, of YAP protein. Furthermore, YAP represents a new drug target for developing therapeutics against cancer. The observation disclosed herein is supported by the report that YAP is enriched in stem cells, although the functional significance of this enrichment is unclear (Ramalho-Santos et al., Science 298: 596-600 (2002)). These studies show that the ability of YAP to promote cell proliferation and prohibit apoptosis might underlie the requirement of YAP in stem cells. Thus, YAP represents a new target for stem cell-based therapies.

The present inventor and others recognized that hpo, sav and wts (also called lats) were identified from genetic screens in Drosophila for negative regulators of tissue growth (Xu et al., 1995; Justice et al., 1995; Tapon et al., 2002; Kango-Singh et al., 2002; Harvey et al., 2003; Wu et al., 2003; Jia et al., 2003; Udan et al., 2003; Pantalacci et al., 2003). Inactivation of any of these genes results in increased cell proliferation and reduced apoptosis. hpo encodes a Ste-20 family protein kinase, sav encodes a protein containing WW and coiled-coil domains, and wts encodes a NDR (nuclear Dbf-2 related) family protein kinase. Studies from several groups and observations by the inventor have suggested that these genes function in a common pathway that coordinately regulates cell proliferation and apoptosis by targeting the cell cycle regulator CycE and the cell death inhibitor DIAP1 (Tapon et al., 2002; Kango-Singh et al., 2002; Harvey et al., 2003; Wu et al., 2003; Jia et al., 2003; Udan et al., 2003; Pantalacci et al., 2003). Using a combination of genetic and biochemical assays, the present inventor has shown that Hpo, Sav and Wts define a protein kinase cascade wherein Hpo, facilitated by Sav, phosphorylates Wts (Wu et al., 2003).

It was further demonstrated that this pathway, hereafter referred to as the Hpo signaling pathway, negatively regulates the transcription of diap] (Wu et al., 2003). It is worth noting that the model differs significantly from an alternative model proposed by others suggesting that the Hpo pathway regulates DIAP1 posttranscriptionally through phosphorylation of DIAP1 by Hpo (Tapon et al., 2002; Harvey et al., 2003; Pantalacci et al., 2003). While the molecular details of this emerging pathway are yet to be completely understood, several lines of evidence have implicated a conserved Hpo signaling pathway in tumorigenesis in mammals. For example, mice lacking a wts homologue develop soft-tissue sarcomas and ovarian tumors (St John et al., 1999). A human ortholog of wts is downregulated in a subset of soft tissue sarcomas (Hisaoka et al., 2002) and the human ortholog of sav is mutated in several cancer cell lines (Tapon et al., 2002). Moreover, the human homologues of wts and hpo could rescue the respective Drosophila mutants (Wu et al., 2003; Tao et al., 1999).

Previous studies of Wts/Lats proteins in Drosophila and mammals have identified a number of putative targets for this important tumor suppressor protein, including the G2/M regulator cdc2, and the actin polymerization regulators zyxin and LIMK1 (Tao et al., 1999; Hirota et al., 2000; Yang et al., 2004). These putative targets, however, do not account for the excessive overgrowth associated with wts mutant clones. On the other hand, the model of the Hpo signaling pathway predicts the existence of Wts target(s) that might regulate the transcription of cycE and diap1. Moreover, such Wts target(s) must account for the overgrowth phenotype associated with wts mutant clones.

As demonstrated herein, yorkie (yki) is a missing link between Wts and transcriptional regulation. yki encodes the Drosophila orthologue of yes-associated protein (YAP), a transcriptional co-activator in mammalian cells (Yagi et al., 1999; Strano et al., 2001; Vassilev et al., 2001). Yki is required for normal tissue growth and diap1 transcription, and is phosphorylated and inactivated by Wts. Importantly, activation of yki phenocopies loss-of-function mutations of hpo, sav or wts, including elevated transcription of cyclin E and diap1, increased proliferation, defective apoptosis and tissue overgrowth. Taken together, these studies identify an elusive cellular target of the Wts protein kinase and provide further support for the present model implicating the Hpo signaling pathway in transcriptional regulation of diap1.

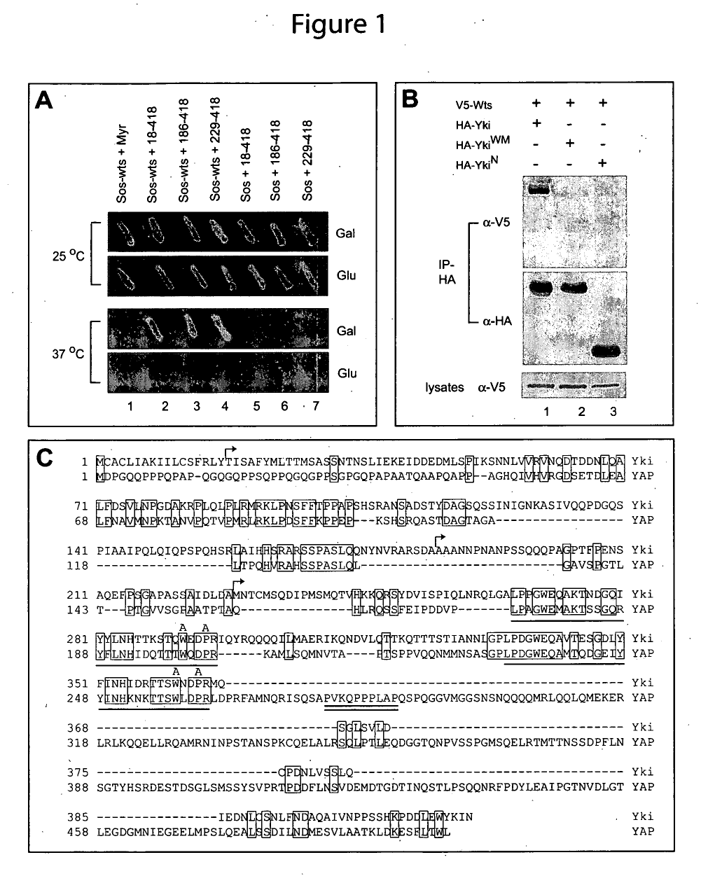

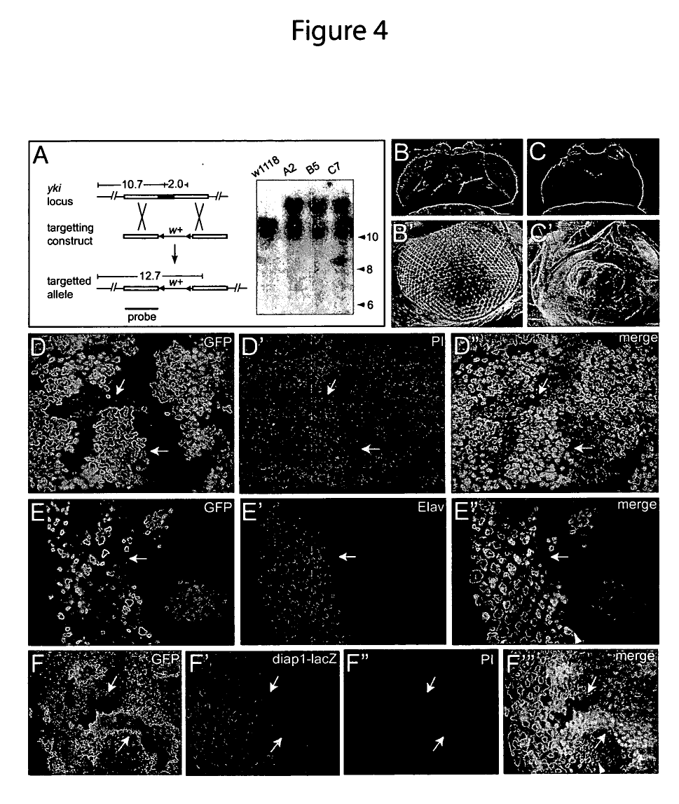

Identification of yki as a Wts-binding protein. Previous studies of Hpo signaling pathway, by the present inventor, placed the Wts protein as the most downstream component among the three tumor suppressors, Hpo, Sav and Wts. In an effort to extend this pathway further downstream, the inventor conducted a yeast two-hybrid screen for Wts-binding proteins. The screen used the Sos recruitment system, which is based on the ability of human Sos protein to complement a temperature sensitive cdc25 allele (cdc25H) in yeast when the Sos protein is targeted to the plasma membrane through bait-prey interactions (Aronheim et al., 1997). In this system, the bait protein is expressed as a fusion protein with human Sos. The prey library contains cDNA clones fused with a myristylation signal that targets proteins to the cell membrane. The expression of the library clones is further controlled by the GAL1 promoter, which is induced in the presence of galactose but repressed in the presence of glucose. When the bait construct and the cDNA library are co-transformed into the cdc25H yeast strain, the only cells capable of growing at restrictive temperature on galactose medium are those that have been rescued by bait-prey interactions that recruit Sos to the cell membrane. Using the non-catalytic N-terminal portion of Wts as bait and from 1 million cDNA clones, 3 independent clones representing partial sequences of the same gene annotated as CG4005 by the Berkeley Drosophila Genome Project (FIG. 1A) were detected. This gene was named yorkie (yki) after Yorkshire Terriers, one of world's smallest breeds of pet dogs, according to its loss-of-function phenotype (see later in FIG. 4C). Consistent with the yeast two-hybrid results, Wts and Yki co-immunoprecipitate with each other when coexpressed in Drosophila S2 cells (FIG. 1B).

The full-length nucleic acid sequence for yki is as follows:

| | |
|---|---|
| AAGTGGACGG GGATAGCCAT CTGGCAACAC TGGGATAAAT TTATTTTATG TTGGCAGTTC | (SEQ ID NO.: 3) |
| CGTAATTATT ATTATTACTA TTATTTATTG CAACGAAGTT TAGTTTTTAA CACCTTAATG | |
| TTATAGTTTC GCGCAGCGCG ATGGAGTGAT ACGTATATGG AAGAACATAT ATGTGCGCGT | |
| GCCTAATCGC TAAGATAATT CTATGTAGTT TTCGTTTGTA TACAATAAGT GCCTTTTATA | |
| TGTTAACGAC GATGTCAGCC AGCAGCAATA CAAACAGCCT GATCGAGAAG GAGATCGACG | |
| ACGAGGACAT GCTTTCGCCG ATCAAGTCCA ACAACCTGGT GGTGCGGGTC AACCAGGACA | |
| CGGACGACAA CCTGCAGGCG CTATTCGACA GCGTCCTGAA TCCGGGTGAC GCCAAGCGCC | |
| CGCTGCAGCT GCCCCTGCGC ATGCGGAAGC TGCCCAACTC CTTCTTCACG CCCCCGGCGC | |
| CCTCGCACTC GCGGGCCAAC AGCGCCGACT CCACCTACGA CGCGGGCTCC CAGTCGAGCA | |
| TCAACATCGG GAACAAGGCG TCCATCGTCC AGCAGCCAGA TGGCCAGTCG CCCATCGCCG | |
| CCATCCCCCA GCTCCAGATT CAGCCGTCTC CCCAGCAGAG CCGCCTGGCG ATACATCACT | |
| CCCGAGCCCG CAGCAGCCCC GCCTCGCTGC AGCAGAACTA CAATGTGCGC GCCCGGAGCG | |
| ACGCAGCAGC AGCCAACAAT CCGAATGCCA ATCCGAGCAG CCAACAGCAG CCCGCTGGGC | |
| CCACTTTCCC AGAGAACAGT GCCCAAGAGT TCCCCAGCGG CGCCCCGGCC AGCTCGGCCA | |
| TTGATCTGGA ACAAATGAAC ACCTGCATGT CGCAGGACAT TCCCATGTCC ATGCAGACAG | |
| TGCACAAGAA GCAGCGCTCC TACGACGTCA TCAGCCCCAT TCAGTTGAAC CGCCAACTAG | |
| GCGCCTTGCC GCCGGGATGG GAGCAAGCCA AGACCAATGA TGGCCAGATC TACTACTTGA | |
| ATCATACTAC AAAATCTACG CAGTGGGAGG ATCCCAGAAT CCAATATCGC CAGCAGCAGC | |
| AAATCTTGAT GGCCGAGCGA ATAAAGCAGA ATGATGTTTT CGAAACTACA AAACAAACTA | |
| CCACATCGAC CATTGCTAAC AATTTGGGTC CACTGCCGGA TGGTTGGGAG CAGGCAGTTA | |
| CCGAGTCCGG AGATCTTTAC TTTATAAATC ACATTGATCG AACGACTTCA TGGAATGATC | |
| CCAGAATGCA ATCTGGGCTT AGCGTGCTCG ACGGCCCAGA TAACTTAGTG TCTTCCCTCC | |
| AGATTGAGGA TAATCTTTGC AGTAACTTGT TCAATGACGC ACAGGCCATT GTAAATCCGC | |
| CGTCTTCCCA CAAACCTGAC GATTTGGAAT GGTATAAAAT TAATTAATTC AATGTATACA | |
| TCTGTATTAG ACCTAAAAGT TTTATATTTT GTATTATTCT AAATTAAATA TTTTTCAAAT | |
| TTTATAGTAT TTCTTTACAT AAAAAAAAAA AAAAAAAAA | |
| Genbank Accession No. DQ99897. | |

yki is most closely related to the human protein yes-associated protein (YAP, also called YAP65) (Sudol, 1994), with 31% identity between the two polypeptides (FIG. 1C). Both proteins contain two WW domains, protein-protein interaction modules composed of 35-40 amino acids that are known to interact with PPXY-containing polypeptides (Macias et al., 2002). The similarity between Yki and YAP extends beyond the WW domains, and includes a stretch of sequence similarity at the N-terminal part of the proteins (FIG. 1C). While initially isolated as a protein that interacts with the SH3 domain of the Yes proto-oncogene, the involvement of YAP in Yes signaling has not been validated (Sudol, 1994). Notably, the corresponding SH3-binding region (Sudol, 1994) is absent in the Drosophila Yki protein (FIG. 1C). On the other hand, YAP has most commonly been implicated as a transcriptional co-activator, a class of transcriptional regulators that do not bind to DNA themselves but associate with DNA-binding transcription factors and supply or stimulate transcriptional activation of the cognate transcription factors. Specifically, YAP has been shown to function as a co-activator for a number of transcription factors, such as the p53 family transcription factor p73 (Strano et al., 2001), the Runt family protein PEBP2α (Yagi et al., 1999) and the TEAD/TEF family transcription factors (Vassilev et al., 2001). However, these studies have been performed exclusively in cultured mammalian cells and little is known about the function of YAP in a physiological context.

The three independent Wts-interacting clones isolated from the yeast two-hybrid screen define the C-terminal half of Yki (residue 229-418) as Wts-binding region (FIG. 1C). This region contains the two predicted WW domains, suggesting that the WW domains are required for the binding between Yki and Wts. Consistent with this hypothesis, a mutant form of Yki carrying mutations of two critical residues of the WW domains abolished the binding between Yki and Wts in Drosophila S2 cells (FIG. 1B). Likewise, the N-terminal half of the Yki protein, which does not contain the WW domains, did not bind to Wts in the same assay (FIG. 1B). Thus, the WW domains of Yki are required for its interaction with Wts.

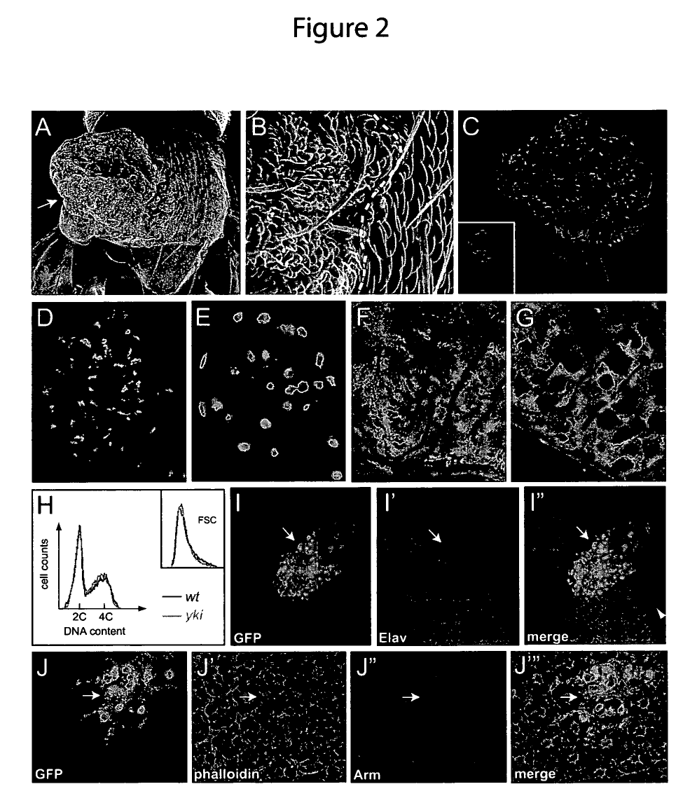

Activation of yki leads to massive tissue overgrowth that resembles the loss-of-function phenotype of hpo, sav or wts. To probe the physiological function of yki, a “flip-out” technique was used to generate clones of cells in which yki is overexpressed during development (Struhl and Basler, 1993; Pignoni and Zipursky, 1997). yki-overexpressing clones led to marked overgrowth in adult epithelial structures (FIG. 2A). When measured by area in two-dimensional images, wing imaginal discs containing multiple yki-overexpressing clones could reach up to 8 times the size of control wing imaginal discs raised under identical conditions (FIG. 2C). This is likely an underestimation of the actual overgrowth due to the highly-folded nature of the mutant disc epithelia (FIG. 2C). Besides the overgrowth phenotype, adult cuticles secreted by yki-overexpressing cells displays an unusual texture. In yki-overexpressing clones on the notum, the apical surface of the epidermal cells is domed such that cell-cell boundaries are visible between adjacent cells, whereas cell boundaries are not visible in the neighboring wild-type tissues (FIG. 2B). Both the overgrowth and the abnormal cell morphology caused by yki overexpression closely resemble those shown previously for hpo and wts mutant cells (Wu et al., 2003; Justice et al., 1995), suggesting that these genes might function in a common genetic pathway.

Cell-doubling time for control and yki-overexpressing cells was measured in the wing imaginal disc by analyzing well-separated flip-out clones 48 hours post clone induction (FIGS. 2D and 2E). The cell-doubling time for wildtype and yki-overexpressing clones (30 pairs of clones analyzed) was 16.1 hrs and 12.0 hrs, respectively. Thus, like mutant clones of hpo or wts, yki overexpressing cells multiply faster in the wing discs. Notably, while cells in the control clones intermingle with their surrounding neighbors to form wiggly borders, yki overexpressing cells minimize their contacts with the surrounding neighbors and form round smooth borders (FIGS. 2D and 2E). Such a phenotype indicates distinct adhesive properties of the yki-overexpressing cells, and resembles that seen with loss-of-function wts clones (FIGS. 2F and 2G). FACS analysis of dissociated wing disc cells showed that yki-overexpressing cells have a similar cell cycle profile and cell size (FSC) distribution as compared to wildtype cells (FIG. 2H). Thus, like loss of function mutations of hpo (Wu et al., 2003), activation of yki does not accelerate a particular phase of the cell cycle. Rather, each phase of the cell cycle is proportionally accelerated.

Activation of yki in the eye imaginal disc leads to increased number of interommatidial cells without affecting photoreceptor differentiation. For the rest of this study, observations were focused on the eye imaginal disc, a pseudostratified epithelium in which cell differentiation, proliferation and apoptosis occurs in a highly stereotyped manner (Wolff and Ready, 1993; Brachmann and Cagan, 2003; Baker, 2001). In the third instar, a morphogenetic furrow (MF) traverses the eye imaginal disc from posterior to anterior. Cells anterior to the MF are undifferentiated and divide asynchronously, whereas cells in the MF are synchronized in the GI phase of the cell cycle. Posterior to the MF, cells either exit the cell cycle and differentiate, or undergo one round of synchronous division (second mitotic wave, SMW) before differentiation. These cells assemble into approximately 750 ommatidia, leaving behind approximately 2000 superfluous cells that are eliminated by a wave of apoptosis ˜36 hr after puparium formation (APF).

To investigate whether activation of yki perturbs cell differentiation, the eye imaginal discs stained for the neuronal marker Elav were examined. As seen in FIGS. 2I-2I″, yki-overexpressing ommatidial clusters have the normal complement of differentiating photoreceptor cells. The spacing between adjacent ommatidial clusters is increased due to the presence of extra interommatidial cells (FIGS. 2I-2I″). The formation of extra interommatidial cells is most evident in pupal eye discs, when yki-overexpressing clones contain many additional cells between photoreceptor clusters (FIGS. 2J-2J″). Thus, as previously seen in hpo, sav or wts mutant clones, yki-overexpressing clones contain an increased number of uncommitted, interommatidial cells without affecting early retina patterning.

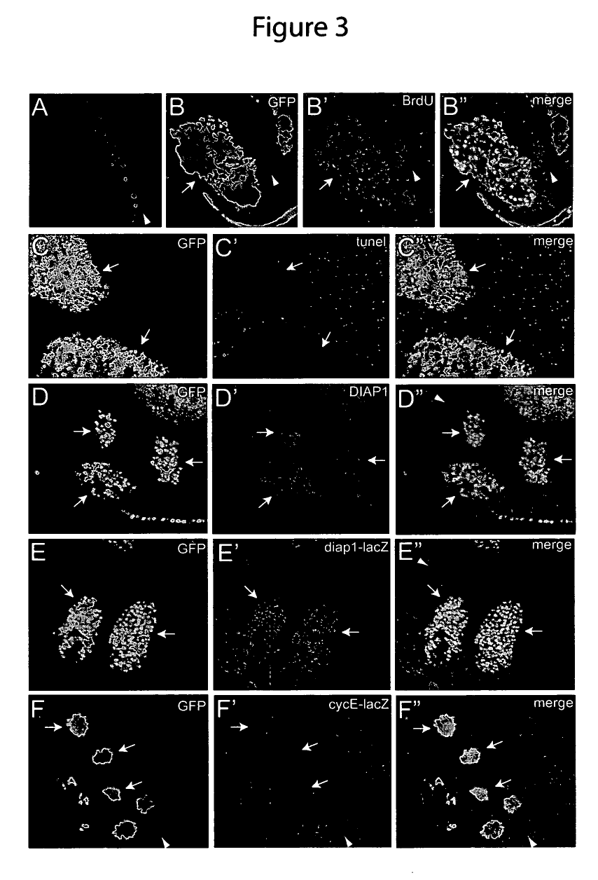

Activation of yki leads to increased cell proliferation and decreased apoptosis. To pinpoint the developmental cause of the yki-overexpression phenotype, cell proliferation and apoptosis in the eye imaginal discs were monitored. In wildtype eye discs (FIGS. 3A), cells are arrested synchronously in GI within the MF, and posterior to the MF, cells in the second mitotic wave (SMW) undergo a synchronous S phase undergo that can be revealed as a band of BrdU-positive cells. Few BrdU-positive cells are found posterior to the SMW. In yki-overexpressing clones, cells fail to undergo cell cycle arrest posterior to the SMW, and continue S-phase (FIGS. 3B-3B″). At least some of these cells continue to proliferate during early pupal development, as revealed by ectopic M phase marker phosphor-histone H3 (PH3) (data not shown) at 16 hr APF.

Developmental apoptosis is most prominent in the pupal retina around 36 hr APF when a wave of apoptosis removes excess interommatidial cells ( Wolff and Ready, 1993; Brachmann and Cagan, 2003; Baker, 2001). Cell death in yki-overexpression clones was monitored using the TUNEL assay. Strikingly, in pupal eyes at 36 hr APF, cell death was significantly suppressed in yki-overexpressing clones, even though abundant apoptosis was detected in the neighboring wildtype cells (FIGS. 3C-3C″). Thus, normal developmental cell death is largely inhibited by yki overexpression.

Activation of yki leads to increased transcription of diap1 and CycE. The increased cell proliferation and decreased apoptosis resulting from yki overexpression are strikingly similar to those caused by loss-of-function mutations in hpo, sav or wts. These observations, together with the identification of Yki as a Wts-binding protein, strongly suggest that Yki functions in the Hpo signaling pathway. To further explore this possibility, the transcription of cell death inhibitor diap1 and cell cycle regulator cycE was examined, known targets of the Hpo signaling pathway (Wu et al., 2003). Elevated DIAP 1 protein was detected in yki-overexpressing clones in the eye imaginal discs (FIGS. 3D-3D″). This regulation is largely mediated at the level of diap1 transcription, since the expression of the th^{j5c8}, a P[lacZ] enhancer trap reporter inserted into the diap1 locus, was similarly elevated in yki-overexpressing clones in a cell-autonomous manner (FIGS. 3E-3E″). Also examined was cycE transcription using a cycE-lacZ reporter that contains 16.4 kb of 5′ regulatory sequence of cycE (Jones et al., 2000). Expression of the cycE-lacZ reporter was increased in yki-overexpressing clones, especially in clones close to the MF (FIG. 3F-3F″), although the effect was less profound than that observed with the diap1 reporter. Thus, like inactivation of hpo, sav or wts, activation of yki results in increased transcription of diap1 and cycE. It is worth noting that the previous analyses of hpo mutant clones also revealed a “tighter” regulation of diap1: while diap1 transcription is elevated in all hpo mutant cells irrespective of their relative position to the MF, cycE transcription is only elevated in hpo mutant cells close to the MF (Wu et al., 2003). These observations suggest that diap1 might represent a more direct transcriptional target of the Hpo signaling pathway.

yki is required for tissue growth and normal diap1 transcription. The overexpression results presented above implicate Yki as a new component of the Hpo signaling pathway, acting antagonistically to Hpo, Sav or Wts. To further explore the role of Yki in the Hpo pathway, a null mutation of yki was generated using the gene targeting strategy developed by Golic and co-workers (Rong and Golic, 2000; Gong and Golic, 2003). The targeting construct was designed in such a way that all the coding sequence of yki was replaced by the w⁺ marker, thus resulting in a complete knockout of gene function (FIG. 4A; see Experimental Procedures for details). yki null mutants are homozygous lethal die at late embryonic and early first instar larval stage. A full-length yki cDNA driven by the ubiquitous α-tubulin promoter completely rescued the yki mutant animals to viable and phenotypically normal adult flies.

To investigate the requirement for yki in tissue growth, the eyeless-FLP technique was used to selectively remove yki function in over 90% of the eye disc cells (Newsome et al., 2000). Eyes composed predominantly of yki mutant cells were markedly reduced in size when compared to control animals (FIGS. 4B-4B′, 4C-4C′). This pinhead phenotype reveals an essential function for yki in tissue growth. To follow yki mutant cells during development, the FLP/FRT system was used to examine genetically marked clones of yki mutant cells. yki mutant clones generated at 40 hr AED were rarely recovered in third instar wing imaginal discs (FIG. 4D-D″). The rare clones recovered contained only a few cells (FIG. 4D-D″). yki mutant clones generated at a similar stage were more frequently recovered in third instar eye discs, but contained much fewer cells when compared to their wildtype twin spots (FIG. 4F). Despite the severe growth defects, loss of yki did not perturb early retina differentiation, as shown by the normal expression of the neuronal marker Elav in yki mutant clones (FIG. 4E). These observations reveal a specific requirement for yki in tissue growth and support an antagonistic relationship between Hpo, Sav or Wts.

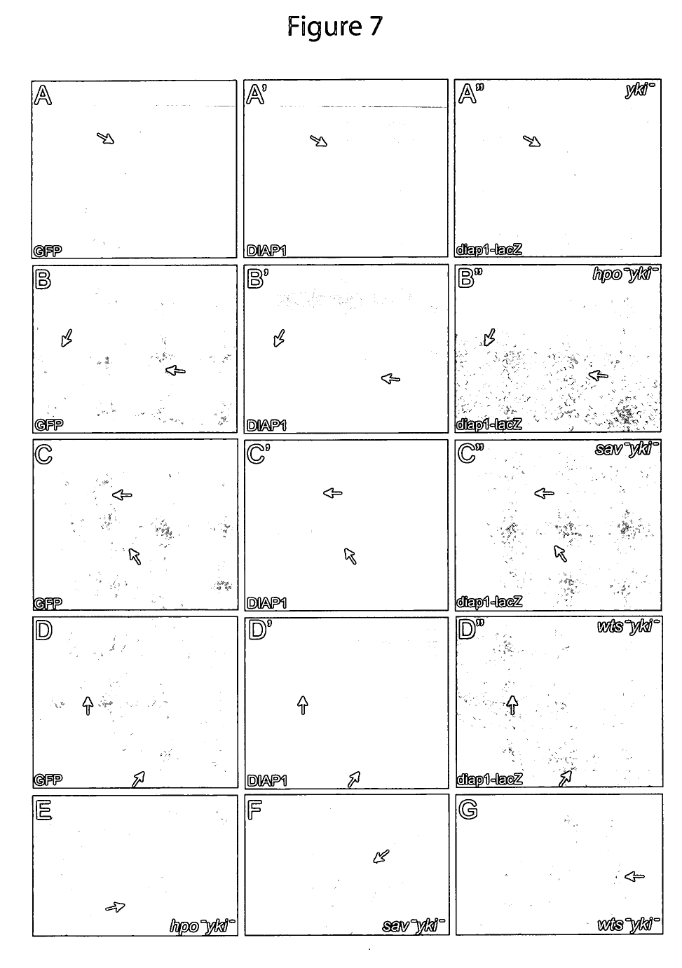

To further probe the requirement of Yki in the Hpo signaling pathway, diap1 transcription in yki loss-of-function mutant clones was determined using the th^{j5c8} diap1-lacZ reporter. Consistent with the overexpression results (FIG. 3), diap1-lacZ expression was reduced in yki mutant cells in a cell autonomous manner (FIG. 4F). Similar results were seen in the wing imaginal discs (data not shown). DIAP1 protein level was also reduced in a cell autonomous manner in yki mutant clones (see below in FIGS. 7A-7A″). Thus, consistent with the observation that yki overexpression upregulates diap1 transcription, yki is required for the normal level of diap1 transcription in Drosophila imaginal discs.

The transcriptional co-activator activity of Yki is negatively regulated by the Hpo pathway. The results presented so far are consistent with a model wherein Yki acts antagonistically to Hpo, Sav and Wts in a common signaling pathway that coordinately controls cell proliferation and apoptosis. Based on the physical interactions between Yki and Wts (FIG. 1), and given that YAP, the mammalian homologue of Yki, is known to function as a transcriptional co-activator (Yagi et al., 1999; Strano et al., 2001; Vassilev et al., 2001), it was hypothesized that Yki functions downstream of Wts to regulate transcription of genes such as diap1, and that the Hpo pathway negatively regulates the co-activator activity of Yki.

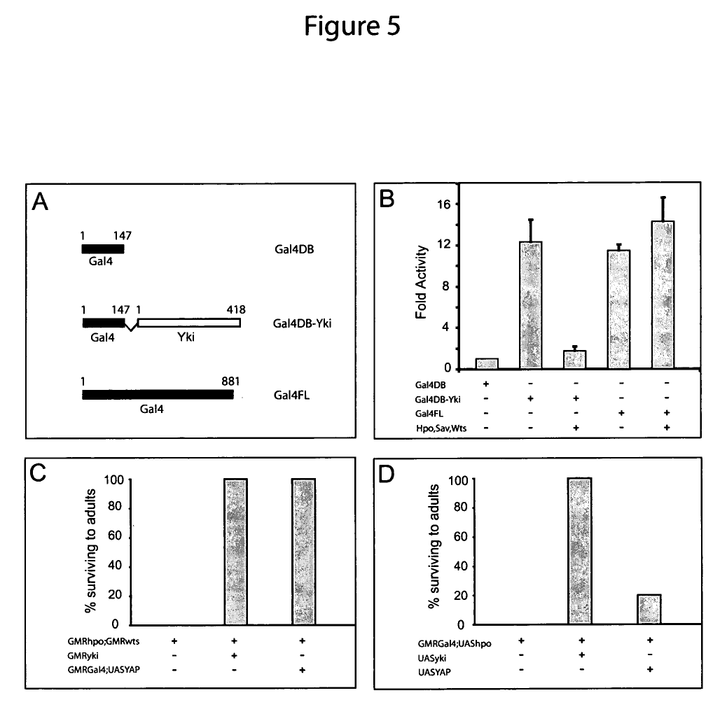

To test that the co-activator activity of Yki is negatively regulated by the Hpo signaling pathway, a transcription assay for Yki activity was established in Drosophila S2 cells. Since the cognate transcription factor(s) that partner with Yki are not yet identified, Yki was fused to the DNA binding domain (DB) of the yeast Gal4 transcription factor (FIG. 5A). The activity of this fusion construct was then assayed using a Gal4-responsive luciferase reporter. Consistent with previous studies of YAP as a transcriptional co-activator in mammalian cells, the Gal4DB-Yki fusion protein exhibited potent transcriptional activation (FIG. 5B). Strikingly, transcriptional activity of the Gal4DB-Yki fusion protein was abolished when Hpo-, Sav- and Wts-expressing plasmids were co-transfected (FIG. 5B). This effect is specific to Yki, since transcriptional activity of the full-length Gal4 (with its own activation domain) was unaffected in the presence of Hpo, Sav and Wts (FIG. 5B). Taken together, these results support the model that the Hpo signaling pathway negatively regulates the co-activator activity of Yki.

Dosage-sensitive genetic interactions between yki and the Hpo signaling pathway. To further probe a functional link between yki and the Hpo pathway, their genetic interactions were determined. Expression of hpo or wts directly from the GMR promoter results in viable flies with rough or slightly rough eyes, respectively, co-introduction of GMR-hpo and GMR-wts into the same animals results 100% lethality at early pupal stage (Wu et al., 2003). Strikingly, such lethality was completely rescued by coexpression of yki resulting from a GMR-yki transgene (FIG. 5C). Interestingly, this lethality was also completely rescued by coexpression of the human YAP gene (FIG. 5C). In another line of studies, the complete pupal lethality caused by the overexpression of UAS-hpo driven by the GMR-Gal4 driver (FIG. 5D) was determined. Interestingly, this lethality was also rescued by the expression of yki (100% rescue) or YAP (21% rescue) (FIG. 5D). Taken together, the genetic interactions described here further support the model that Yki acts antagonistically to Hpo, Sav and Wts in a common signaling pathway. The ability of a human YAP transgene to rescue the lethality of flies caused by Hpo pathway hyperactivation reveals a functional conservation between Yki and YAP, and raises the intriguing possibility that YAP might play a similar role in mammalian growth control.

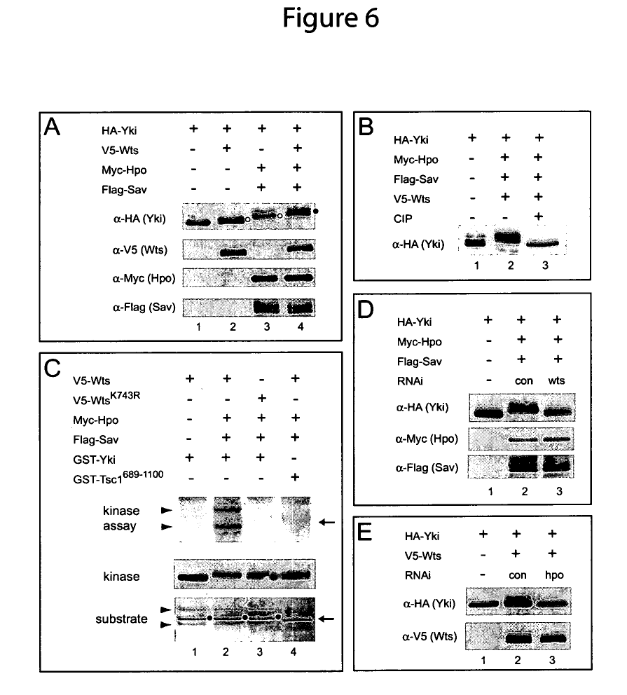

Phosphorylation of Yki by Wts. The observations presented so far are consistent with the model that the Hpo signaling pathway negatively regulates the activity of Yki. Given that Wts directly associates with Yki (FIG. 1) and that Wts encodes a protein kinase, the simplest model is that Yki is regulated by Wts through direct protein phosphorylation. The phosphorylation of Yki by the Hpo signaling pathway was determined using an S2 cell-based assay. As shown in FIG. 6A, co-expression of Wts and Yki resulted in a small mobility retardation of Yki (compare lanes 1 and 2). Co-expression of Hpo-Sav with Yki also resulted in a mobility retardation of Yki (compare lanes 1 and 3), and co-expression of Hpo-Sav-Wts with Yki resulted in an even greater mobility shift of Yki (compare lanes 1-4). The mobility shift of Yki induced by Hpo-Sav-Wts expression was abrogated by phosphatase treatment, suggesting that this mobility shift is due to protein phosphorylation (FIG. 6B). It is worth noting that the increasing phosphosphorylation of Yki induced by Wts, Hpo-Sav and Hpo-Sav-Wts in the S2 cell assay (FIG. 6A) correlates with the severity of the overexpression phenotype resulting from the respective transgenes in vivo: expression of Wts by the. GMR promoter results in slightly rough eyes, expression of Hpo-Sav results in strong rough eyes with reduced size, and expression of Hpo-Sav-Wts results in complete animal lethality (Tapon et al., 2002; Wu et al., 2003). Taken together, these results suggest that Yki phosphorylation is a relevant output of the Hpo signaling pathway.

To determine whether Yki is a direct substrate of Wts, in vitro kinase assays were conducted using immunoprecipitated V5-tagged Wts protein expressed in S2 cells as the kinase source and GST-Yki fusion protein as substrate. When expressed alone, Wts had little kinase activity on the Yki substrate (FIG. 6C, lane 1). When co-expressed with Hpo-Sav, however, Wts displayed specific kinase activity towards Yki (FIG. 6C, lane 2) but not a control substrate (FIG. 6C, lane 4). The effect of Wts on Yki is direct, as a kinase dead mutation of Wts, WtsK⁷⁴³R, completely abolished the in vitro kinase activity of Wts towards Yki (FIG. 6C, lane 3). These data confirm that Yki is a kinase substrate of Wts. Furthermore, the observation that Hpo-Sav stimulate the in vitro kinase activity of Wts towards Yki is consistent with the previous report showing activation of Wts by Hpo-Sav as measured by Wts phosphorylation in an S2 cell assay (Wu et al., 2003).

It was next determined if Wts is the kinase that directly phosphorylates Yki by transfection of Hpo-Sav lead to mobility shift of Yki in the S2 cell assay (FIG. 6A). It was also determined, if the kinase activity of Wts towards Yki requires activation by Hpo-Sav, why did transfection of Wts alone lead to mobility shift of Yki in the same assay (FIG. 6A). It is reasoned, but in now way a limitation of this invention, that the mobility shift induced by transfected Hpo-Sav or Wts in the S2 cell assay might require the endogenous Wts or Hpo, respectively. Indeed, RNAi of wts completely reversed the mobility shift of Yki induced by Hpo-Sav expression (FIG. 6D), and RNAi of hpo completely reversed the mobility shift of Yki induced by Wts expression (FIG. 6E). These data further support the model that Yki is phosphorylated by Wts upon activation of the Hpo signaling pathway.

yki is genetically epistatic to hpo, sav and wts. The genetic evidence presented so far suggest that yki acts antagonistically to hpo, sav and wts. The biochemical studies further refined this model and demonstrate that Yki is phosphorylated and inactivated by the Hpo signaling pathway via direct phosphorylation by Wts. A prediction of this molecular model is that loss-of-function mutations of yki should be genetically epistatic to those of hpo, sav or wts. To test this hypothesis, clones of cells were generated that are doubly mutant for hpo-yki, sav-yki or wts-yki using the FLP/FRT system. Since yki and sav (or wts) are located on different chromosome arms (2R for yki and 3R for sav or wts), sav-yki and wts-yki double mutant clones were generated using insertions of the tubulin-yki rescue construct on 3R in a yki null mutant background. Consistent with the molecular model that places Yki downstream of Hpo, Sav and Wts, hpo-yki, sav-yki or wts-yki double mutant clones displayed phenotypes indistinguishable from those of yki single mutant clones, including retarded growth, decreased DIAP1 protein levels and decreased diap1 transcription (FIG. 7). These genetic observations further strengthen the molecular model implicating Yki as a direct target of Wts in the Hpo signaling pathway.

The mechanisms of how body and organ size are regulated are just beginning to be understood (reviewed by Conlon and Raff, 1999; Hipfner and Cohen, 2004). Recent studies in Drosophila have implicated a number of pathways in the coordinate control of cell growth, proliferation and apoptosis, which ultimately regulate body and organ size. The insulin/Tsc/TOR signaling network, for example, plays a major role in coordinating organ growth with environmental cues such as nutrients (reviewed by Hafen, 2004; Pan et al., 2004). The Hpo signaling pathway, on the other hand, might contribute to an intrinsic size “checkpoint” that normally stops growth when a given organ reaches its characteristic size (reviewed by Hay and Guo, 2003; Ryoo and Steller, 2003). Thus, molecular elucidation of the Hpo signaling pathway should provide important insights into size-control mechanisms in development and tumorigenesis.

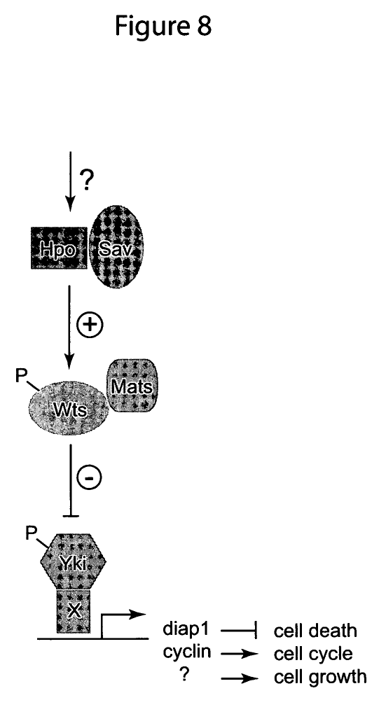

Identification of Yki as a direct target of the Wts/Lats protein kinase in the Hpo signaling pathway. While previous studies have implicated a number of proteins, including cdc2, zyxin and LIMK1, as putative targets of the Wts/Lats kinase, none of these targets could account for the overgrowth seen in wts mutant clones in Drosophila (Tao et al., 1999; Hirota et al., 2000; Yang et al., 2004). Thus, the most critical target of the Wts/Lats kinase has remained elusive. In this study, genetic and biochemical studies demonstrate that Yki, the Drosophila orthologue of the mammalian co-activator protein YAP, as a direct, critical target of the Wts/Lats kinase in the Hpo signaling pathway (FIG. 8). First, Yki associates with Wts both in yeast and Drosophila cells. Second, Yki is phosphorylated by Wts, both in a cell culture assay and in vitro using immunoprecipitated Wts protein. In both cases, Wts-mediated phosphorylation of Yki is stimulated by upstream components of the Hpo pathway, providing further support for the significance of this phosphorylation. Third, the extent of Yki phosphorylation induced by Hpo pathway components in the S2 cell assay (FIG. 6A) correlates with the severity of the overexpression phenotype caused by the respective transgenes in vivo. Fourth, the transcriptional co-activator activity of Yki is antagonized by the Hpo signaling pathway. Fifth, overexpression of yki phenocopies loss-of-function mutations of hpo, sav or wts, including elevated transcription of cyclin E and diap1, increased proliferation, defective apoptosis and tissue overgrowth. Lastly, yki is required for tissue growth and normal diap1 transcription, and the epistasis analyses unambiguously placed yki genetically downstream of hpo, sav and wts. Taken together, these observations provide compelling evidence that Yki is a critical target of the Wts/Lats tumor suppressor protein in the Hpo signaling pathway. The relationship between Yki and Hpo signaling is likely conserved during evolution, since overexpression of mammalian YAP was able to rescue the lethality associated with hyperactivation of the Hpo pathway in Drosophila (FIG. 5). The functional conservation between Yki and YAP also raises the possibility that YAP might function as an oncogene in mammals.

While little is known about how the activity of the Hpo signaling pathway is regulated in vivo, the loss of hpo, sav or wts, or overexpression of yki, delay cell cycle exit at late stages of imaginal disc development. The components of this pathway show temporal changes in activity during imaginal disc growth.

Yki represents the first known substrate of any NDR family kinase. Besides the Wts/Lats proteins, the NDR family include Cbk1, Dbf2 and Dbf20 in budding yeast; Sid2 and Orb6 in fission yeast; Cot-1 in Neurospora; Sax-1 in C. elegans; Trc in Drosophila; and NDR1 and NDR2 in mammals (reviewed by Tamaskovic et al., 2003). While the physiological functions of mammalian NDR1 and NDR2 are yet to be demonstrated, other NDR family kinases have been implicated in diverse events in cell cycle and cell morphogenesis. For example, Cbk1 is required for maintaining polarized growth during budding and mating as well as cell wall remodeling after cytokinesis. Dbf2 and Dbf20 are part of the mitotic exit network (MEN) that coordinates CDK inactivation and cytokinesis. In fission yeast, Sid2 functions in the septation initiation network (SIN), a fission yeast counterpart of the budding yeast MEN, while Orb6 is maintains cell polarity during cell cycle progression. The Cot-1 protein of Neurospora is required for hyphal outgrowth, while Sax-1 of C. elegans and Trc of Drosophila regulates neuronal morphogenesis and polarized cell extensions, respectively. Despite their diverse cellular functions, all NDR family kinases share similar structural features, such as the insertion of 30-60 amino acids between the kinase subdomains VII and VIII, the presence of conserved activation loop and hydrophobic motif, and the presence of N-terminal non-catalytic domain (reviewed by Tamaskovic et al., 2003). These common features suggest that NDR family kinases may employ similar mechanisms to interact with their substrates and regulators. The basic approach described herein, viz., using the N-terminal non-catalytic domain of Wts as yeast two-hybrid bait to identify its substrate, represents a general method to discover substrates for other NDR family kinases.

Regulation of NDR family kinases by Ste-20 family kinases. The Hpo signaling pathway includes a model whereby Hpo, somehow facilitated by Sav, phosphorylates Wts (Wu et al., 2003). The identification of Yki as a Wts substrate provides a new tool to evaluate the earlier model. Consistent with this model implicating Hpo as an upstream kinase that activates Wts, it is demonstrated herein that in Drosophila S2 cells, the mobility shift of Yki induced by Wts is dependent on the endogenous Hpo protein (FIG. 6E). Conversely, the mobility shift of Yki induced by Hpo-Sav is dependent on the endogenous Wts protein (FIG. 6D). Furthermore, the in vitro kinase activity of Wts towards Yki is strongly stimulated when Wts is co-expressed with Hpo-Sav, suggesting that such a relationship between Hpo and Wts is likely conserved during evolution. Indeed, a recent study has demonstrated the activation of the mammalian Lats1 kinase by the mammalian Hpo homologues Mst1/Mst2 (Chan et al., 2005).

It is worth noting that several Ste-20 family proteins have been implicated as upstream activating kinases for NDR kinases. Such examples include the activation of Wts by Hpo (Wu et al., 2003; Chan et al., 2005), the activation of Dbf2 by Cdc15 (Mah et al., 2001), the regulation of Orb6 by Pak1 (Verde et al., 1998), and the regulation of Sid2 by Sid1 (Guertin et al., 2000). Thus, activation by Ste-20 family kinases might represent a general mechanism for regulating NDR kinases. In retrospect, the difficulties in identifying substrates for NDR kinases might be due to their stringent substrate specificity in conjunction with a requirement for activation by upstream kinases. Another emerging feature of the NDR kinases concerns their regulation by the Mob family of small regulatory proteins, which have been found to associate with multiple NDR family kinases, such as Dbf2, Orb6, Sid2 and Cbk1 in yeast, as well as NDR1 and NDR2 in humans (reviewed by Tamaskovic et al., 2003). In the best studied example, it was shown that Mob1 facilitates the phosphorylation of Dbf2 by Cdc15 at Ser-374 and Thr-544 residues in the activation loop and hydrophobic motif, respectively (Mah et al., 2001). In Drosophila, Mats, a Mob family protein, has recently been identified as a tumor suppressor gene that likely regulates Wts in the Hpo signaling pathway (Lai et al., 2005). Mats associates with Wts and was proposed to act as an activating subunit of Wts kinase based on its stimulation of Wts autophosphorylation. However, the relationship between Wts activation by Mats and Hpo-Sav was not explored (Lai et al., 2005). In light of results from previous studies of Mob1 and Dbf2 (Mah et al., 2001), stimulation of Wts autophosphorylation by Mats in the IP kinase assay could also be explained by Mats functioning upstream of Wts and facilitating its activation by endogenous Mst1/Mst2 proteins in 293T cells. Therefore, the techniques disclosed herein may be used to distinguish whether Mats activates the kinase activity of Wts per se or facilitates the activation of Wts by upstream Hpo pathway components. Irrespective of its molecular mechanism, regulation by Mob family proteins likely represents an important and shared feature of modulating NDR family kinases.

Transcriptional regulation of diap1 by the Hpo signaling pathway. Previous studies of the Hpo signaling pathway suggested two contrasting models on how this pathway regulates the cell death regulator DIAP1. Using a diap1-lacZ reporter to follow diap1 transcription, elevated diap1 transcription was observed in mutant clones of hpo, sav or wts that closely matches the increase in DIAP1 protein levels. Based on these observations, the Hpo pathway negatively regulates diap1 at the level of transcription (Wu et al., 2003). Meanwhile, an alternative model suggested that Hpo regulates DIAP1 posttranscriptionally by directly phosphorylating DIAP1, thus promoting its degradation (Tapon et al., 2002; Harvey et al., 2003; Pantalacci et al., 2003). This model was largely based on two lines of evidence, including in situ hybridization showing unchanged diap1 mRNA level in mutant clones and the ability of Hpo to phosphorylate DIAP1 in vitro. In situ hybridization used in the latter studies did not involve the marking of mutant clones and thus maybe less definitive than the diap1-lacZ reporter. A major drawback of the posttranscriptional model is that it can not account easily for the involvement of Wts in the Hpo pathway. A direct link between Hpo and DIAP1 inevitably implies Wts as acting upstream or in parallel with Hpo, which is contradictory to other studies of the NDR kinases that generally place them downstream of the Ste2O kinases (Wu et al., 2003; Chan et al., 2005; Mah et al., 2001; Verde et al., 1998; Guertin et al., 2000).

If the Hpo signaling pathway regulates diap1 via a transcriptional mechanism, then one or more transcriptional regulator(s) should exist that control diap1 transcription whose activity maybe regulated by the Hpo signaling pathway. Furthermore, such transcriptional regulator(s) must account for the mutant phenotypes resulting from deregulation of the Hpo pathway. As demonstrated herein, Yki represents such a transcriptional regulator. YAP, the mammalian homologue of Yki, acts as a transcriptional co-activator in mammalian cells (Yagi et al., 1999; Strano et al., 2001; Vassilev et al., 2001). Yki is shown to be required for normal diap1 transcription in Drosophila imaginal discs and that overexpression of Yki results in elevated diap1 transcription. In Drosophila S2 cells, fusion of Yki with the Gal4 DNA binding domain confers transcriptional activation that is in turn antagonized by hyperactivation of the Hpo pathway. Importantly, overexpression of Yki recapitulates the mutant phenotypes of hpo, sav and wts, including changes in gene transcription at the molecular level, defects in cell proliferation and apoptosis at the cellular level, as well as excess overgrowth at the tissue level. Taken together, these results identify Yki as a major effector of the Hpo pathway and further support the previous model implicating the Hpo signaling pathway in transcriptional regulation of diap1.

Understanding the molecular mechanisms by which the Hpo signaling pathway regulates diap1 transcription will provide important insights into the developmental coordination of tissue growth and apoptosis. Like other transcriptional co-activators, Yki presumably functions by interacting with DNA-binding transcription factor. YAP, the mammalian homologue of Yki, is known to function as co-activator for a number of transcription factors, such as the p53 family member p73 (Strano et al., 2001), the Runt family member PEBP2α (Yagi et al., 1999) and the four TEAD/TEF transcription factors (Vassilev et al., 2001). This interaction is generally mediated by the WW domains of YAP and the PPxY motifs of the cognate transcription factors. Interestingly, the PPxY motifs are present in many transcription factors, suggesting that interactions between WW domains and PPxY motifs might play a more general role in transcriptional activation (Yagi et al., 1999).

It is worth noting that while the reported ability of YAP to transactivate p73 in cultured mammalian cells is more suggestive of a tumor suppressor function for YAP (Basu et al., 2003), these studies clearly implicate Yki and YAP as oncogenes. One interesting possibility is that the reported coupling of mammalian YAP to p73 might represent a fail-safe mechanism to limit the oncogenic potential of YAP in much the same way as cell death is obligatorily linked to activation of Myc and Ras (reviewed by Lowe et al., 2004). An important direction in the future is to identify the DNA binding transcription factor (denoted as “X” in FIG. 8) that partners with Yki to coordinately regulate the transcription of cell cycle and cell death genes, which should provide critical insights into how Yki (and likely YAP as well) could function as a potent oncogene. This effort will be facilitated by the dissection of the diap1 promoter and the identification of a minimal Hpo responsive element (HRE) that confers transcriptional regulation of diap1 by the Hpo signaling pathway. With such a DNA element, one should be able to identify the cognate DNA binding transcription factor that partners with Yki to regulate the transcription of diap1 and other Hpo-pathway responsive genes.

A conserved role for the Hpo signaling pathway in mammalian growth control and tumorigenesis. Many components of the Hpo signaling pathway are highly conserved between Drosophila and mammals, suggesting that this emerging pathway might play a similar role in mammals. Indeed, previous studies have shown that human homologues of wts, hpo and mats could rescue the respective Drosophila mutants (Wu et al., 2003; Tao et al., 1999; Lai et al., 2005). Moreover, mice lacking a wts homologue are prone to tumor formation (St John, et al., 1999), and the human orthologs of sav and mats are mutated in several cancer cell lines (Tapon, et al., 2002; Lai, et al., 2005). Such conservation is further extended by the present observation that the human homologue of Yki, the newest component of the Hpo pathway, has similar biological activity as its fly counterpart when assayed in Drosophila. Taken together, it was found herein that the Hpo signaling pathway is likely to play a conserved role in mammalian growth control. Furthermore, loss-of-function mutations in tumor suppressors of the Hpo pathway, including homologues of Hpo, Sav, Wts and Mats, and gain-of-function mutations in oncogenes of this pathway such as YAP, are likely to contribute to mammalian tumorigenesis.

All known components of the Hpo pathway are conserved from flies to humans. These include Hpo (MST1 and MST2 in mammals), Sav (hWW45 in mammals), Wts (Lats1 and Lats2 in mammals) and Yki (YAP in mammals). Functionally, the present inventors have shown that a human MST2 gene can rescue the hpo mutant flies (Wu et al., 2003). Furthermore, the Drosophila Yki protein and its human homologue YAP have similar biological activities in flies (Huang et al., 2005). These results raise the possibility that the Hpo signaling pathway might play a conserved role in mammalian growth control, although this hypothesis has not been systematically examined.

To determine the role of the Hpo signaling pathway in mammalian growth control, a mouse liver model was used as our model organ because liver has long been used as a model system to study mammalian organ size control, ranging from the ability of liver to regenerate its full size after hepatectomy to the ability of oncogenes to induce liver hyperplasia and liver tumors (Michalopoulos and DeFrances, 1997). Specifically, transgenic mice were generated to express the human YAP gene specifically in the liver in a tetracycline-inducible manner. This was achieved using the liver-specific ApoE-rtTA (rtTA: reverse tetracycline-controlled transactivator) together with TRE (Tet-responsive element)-YAP. Transgenic mice harboring these two elements allow the YAP gene to be expressed in the liver in a tetracycline-dependent manner. If the YAP gene plays a conserved role in organ size control as its Drosophila counterpart, it was recognized herein that expression of YAP might result in an increase in organ size. Indeed, it was found that expression of the YAP gene (by feeding the mice with Doxycycline-containing water) results in massive liver overgrowth (data not shown). Thus, like its Drosophila homologue (Yki), the mammalian YAP gene functions as a potent oncogene to promote organ growth. This transgenic mouse study demonstrates that the Hpo signaling pathway plays an important role in mammalian growth control.

It is worth noting that the oncogenic activity of YAP discovered by us is exactly opposite to what has been reported about YAP in the literature. In mammalian cell culture, YAP has been previously reported to activate p73 and enhance apoptosis (Basu et al., 2003). This would imply, that YAP functions as a tumor suppressor gene. To the contrary, our studies of Yki and YAP clearly show that these genes function as oncogenes, not tumor suppressors. Since our experiments are carried out in vivo (in both flies and mice), we believe that our experiments reflect the true physiological function of the Yki and YAP genes.

Gene targeting and rescue of yki. An ends-out, or replacement, gene targeting strategy developed by Golic and co-workers (Gong and Golic, 2003) was used to generate a null mutation in yki. This strategy involves cloning DNA segments flanking target locus into a specially designed targeting vector containing a w^{hs} marker gene between the homologous DNA segments, as well as features that allow the generation of a linear DNA template for homologous recombination with the target locus following the action of FLP site-specific recombinase and I-SceI endonuclease.

Briefly, two pairs of oligos were used:

(SEQ ID NO.:4)

5′-AGCAGGCGCGCCAATGTATACATCTGTATTAGACC-3′
and

(SEQ ID NO.:5)

5′-AGCAGGCGCGCCCTTACAAAACTTTTGCCACTG-3′;
and

(SEQ ID NO.:6)

5′-AGCAGCGGCCGCGGGGTGTTAGTAGCTTCAGGGTT-3′
and

(SEQ ID NO.:7)

5′-AGCAGCGGCCGCATCTTAGCGATTAGGCACGCGCAC-3′,

These oligos were used to amplify DNA fragments of ˜4 kb from the BAC clone 27M17 (Berkeley Drosophila Genome Project). These fragments, representing the left and right homologous arms, were cloned into the AscI and NotI site of pW25, respectively. This targeting construct is expected to replace all the coding sequence of yki (292097-294339 nucleotides of the Drosophila genome locus AE003462) with w^{hs}. Transgenic flies carrying the targeting construct on the 3^{rd} chromosome were crossed to flies carrying the FLP and I-SceI enzyme source, and the progeny were screened for homologous recombination at the target locus by following the movement of the donor element (marked by w^{hs}) from the 3rd chromosome to the 2^{nd} chromosome, followed by molecular verification of gene targeting by Southern blotting. A special feature of the pW25 vector is that the w^{hs} marker is flanked by lox sites thus allowing the removal of w^{hs} marker by expression of Cre recombinase.

The rescue construct of yki was made by cloning a full-length yki cDNA downstream from the α-tubulin promoter (a gift from Jin Jiang, University of Texas Southwestern Medical Center, Dallas, Tex.). Multiple insertion lines of the tub-yki construct on the X and 3^{rd} chromosomes were tested for their ability to rescue three independently derived yki knockout alleles. In all transgene/mutant combinations, the transgenes completely rescued yki knockout alleles to wildtype flies.

Yeast two hybrid screens. Yeast two-hybrid screens were carried out using Stratagene's CytoTrap system or the Sos expression and Drosophila cDNA library according to manufacturer's instructions. The bait plasmid was constructed using an N-terminal fragment of Wts (1-608).

The three independent yki clones recovered from the yeast two-hybrid screen differ in their N-termini, starting at amino acid 18, 186 and 229, respectively. These clones are otherwise identical, all including the termination codon and polyA sequence. The Berkeley Drosophila Genome project has sequenced a cDNA (LD21311) from the yki locus. LD21311 represents a differentially spliced form of yki, and differs from the yeast two-hybrid positives in that it contains only one WW domain at the C-terminal region of the protein. This one-WW form of Yki generates a much weaker overexpression phenotype as compared to the two-WW form (J.H, unpublished observations). To construct a full-length yki cDNA with two WW domains, a BamHI-XhoI fragment of LD12311 containing a one WW domain was replaced with the corresponding fragment from the yeast two-hybrid clones containing two WW domains. This yki cDNA clone was used in all subsequent analyses.

Drosophila strains. The following flies have been described previously: GMR-Hpo (Wu et al., 2003), GMR-Wts (Tapon et al., 2002), hpo⁴²⁻⁴⁷ (Wu et al., 2003), sav³ (Tapon et al., 2002), wts^{X1} (Xu et al., 1995), th^{j5c8} (Hay et al., 1995; Ryoo et al., 2002), cycE-lacZ. (Jones et al., 2000). Transgenic flies overexpressing yki were generated by cloning yki cDNA into the pUAST and pGMR vectors. The human YAP cDNA clone (IMAGE clone 5747370) was obtained from Invitrogen and cloned into the pUAST vector to generate UAS-YAP flies. The eyeless-FLP technique (Newsome et al., 2000) was employed to selectively remove yki activity in the eye imaginal disc, using flies of the genotype: y w ey-flp; FRT42D yki^{B5}/FRT42D w^{+l l(}2)c1-R11.

The FLP/FRT system was used to generate mutant clones in imaginal discs, which were identified by the lack of a ubiquitously expressed GFP marker. For double mutant clones of hpo, sav or wts with yki, the following genotypes were used. Note that a tub-yki rescue construct insertion on 3R was used in generating sav yki and wts yki double mutant clones.

hpo yki double mutant clones:

y w hsp-flp; FRT42D hpo⁴²⁻⁴⁷yki^{B5}/FRT42D Ubi-GFP

sav yki double mutant clones:

y w hsp-flp; FRT42D yki^{B5}/FRT42D yki^{B5}; FRT82B sav³/FRT82B Ubi-GFP P[tub-yki]

wts yki double mutant clones:

y w hsp-flp; FRT42D yki^{B5}/FRT42D yki^{B5}; FRT82B wts^{X1}/FRT82B Ubi-GFP P[tub-yki]

S2 cell culture, immunoprecipitation, in vitro kinase assays. S2 cells were propagated in Drosophila Serum Free Medium (SFM, Invitrogen) supplemented with L-Glutamine and antibiotics. RNAi of S2 cells was carried out as described previously, and dsRNA of the mammalian CYP7A1 gene was used as wildtype control (Gao et al., 2002).

HA-tagged Yki was constructed by adding a C-terminal HA epitope (YPYDVPDYA) to Yki using the pAc5.1/V5-HisB vector (Invitrogen). N-terminal V5-tagged Wts was constructed in the same vector by adding a V5 tag (GKPIPNPLLGLDST) between the first and second codon of Wts. Point mutations of Yki and Wts were introduced using the QuikChange site-directed mutagenesis kit (Stratagene). Myc-tagged Hpo and Flag-tagged Sav constructs have been described previously (Wu et al., 2003). The pGEX4T-1 vector was used to express a fusion of GST with full length Yki the GST-Yki preparation contained two Yki-related bands, with the upper band migrating at the expected molecular weight. It also contained a contaminating band (FIG. 6C). The Yki-related bands, but not the contaminating band, were diminished by thrombin, which cleaves the GST-Yki fusion at the junction of GST and Yki (data not shown). GST-Tsc1 fusion has been described (Wu et al., 2003). Transfection and immunoprecipitation in S2 cells were carried out as described previously (Gao and Pan, 2001). For in vitro kinase assay, S2 cells expressing V5-tagged Wts or Wts^{K743R} were lysed in lysis buffer containing 50 mM HEPES (pH7.4), 50 mM NaCl, 1 mM EDTA, 0.5% NP-40 plus phosphatase and protease inhibitors cocktail. Wts was immunoprecipitated with anti-V5 antibody and protein G-Sepharose. Immunoprecipitates were washed and incubated with recombinant substrate GST fusion proteins in kinase buffer containing 40 mM HEPES (pH7.4), 10 mM MgCl2, 10 μM ATP and 10 μCi/ml γ-P³²ATP at 30° C. for 45 minutes.

Luciferase reporter assays. The pActGal4(1-147)SK vector (a gift of Albert Courey). was used to construct an in-frame fusion of the DNA binding domain (DB) of Gal4 transcription factor and the full-length Yki protein. The resulting plasmid, pActGal4DB-Yki, was transfected into Drosophila S2 cells along with G5-37tkluc, a Gal4-responsive luciferase reporter plasmid (a gift of Albert Courey), with and without plasmids expressing Hpo, Sav and Wts. Triplicates were set up for each transfection. After culturing S2 cells for 48 hours in Drosophila SFM (Invitrogen) supplemented with L-Glutamine, cells were then washed with 1 ml 1× phosphate-buffered saline. Cells were then lysed in 200 μl 1× Passive Lysis Buffer (PLB, Promega). Luciferase measurements were taken by mixing 20 μl lysate and 50 μl Luciferase Assay Reagent II in a 96-well plate. Light intensity was measured a total of 10 seconds at 2 second increments in a FLUOstar luminometer (BMG LabTechnologies).

Cell cycle analysis. FACS analysis of dissociated imaginal wing disc cells was performed as described (Neufeld et al., 1998) using FACStar machine and analyzed with CellQuest program. Clones of cells overexpressing yki were generated using Act>CD2>Gal4, UAS-GFP, and UAS-yki transgenic flies. Clones were induced at 72 hr AED, and analyzed at 120 hr AED. Cell doubling times were derived using the formula (log 2/log N)hr, where N=median number of cells/clone and hr =time between heatshock and disc fixation.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including, ” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

Aronheim,A., Zandi,E., Hennemann,H., Elledge,S. J., and Karin,M. (1997). Isolation of an AP-1 repressor by a novel method for detecting protein-protein interactions. Mol. Cell Biol. 17, 3094-3102.

Basu,S., Totty,N. F., Irwin,M. S., Sudol,M., and Downward,J. (2003). Akt phosphorylates the Yes-associated protein, YAP, to induce interaction with 14-3-3 and attenuation of p73-mediated apoptosis. Mol. Cell 11, 11-23.

Baker,N. E. (2001). Cell proliferation, survival, and death in the Drosophila eye. Semin. Cell Dev. Biol. 12, 499-507.

Brachmann,C. B. and Cagan,R. L. (2003). Patterning the fly eye: the role of apoptosis. Trends Genet. 19, 91-96.

Chan,E. H., Nousiainen,M., Chalamalasetty,R. B., Schafer,A., Nigg,E. A., and Sillje,H. H. (2005). The Ste20-like kinase Mst2 activates the human large tumor suppressor kinase Lats1. Oncogene 24, 2076-2086.

Conlon,I. and Raff,M. (1999). Size control in animal development. Cell 96, 235-244.

Gao,X. and Pan,D. (2001). TSC1 and TSC2 tumor suppressors antagonize insulin signaling in cell growth. Genes Dev. 15, 1383-1392.

Gao,X., Zhang,Y., Arrazola,P., Hino,O., Kobayashi,T., Yeung,R. S., Ru,B., and Pan,D. (2002). Tsc tumour suppressor proteins antagonize amino-acid-TOR signalling. Nat. Cell Biol. 4, 699-704.

Gong,W. J. and Golic,K. G. (2003). Ends-out, or replacement, gene targeting in Drosophila. Proc. Natl. Acad. Sci. U.S.A 100, 2556-2561.

Guertin,D. A., Chang,L., Irshad,F., Gould,K. L., and McCollum,D. (2000). The role of the sidlp kinase and cdc14p in regulating the onset of cytokinesis in fission yeast. EMBO J. 19, 1803-1815.

Hafen,E. (2004). Interplay between growth factor and nutrient signaling: lessons from Drosophila TOR. Curr. Top. Microbiol. Immunol. 279, 153-167.

Harvey,K. F., Pfleger,C. M., and Hariharan,I. K. (2003). The Drosophila Mst ortholog, hippo, restricts growth and cell proliferation and promotes apoptosis. Cell 114, 457-467.

Hay,B. A. and Guo,M. (2003). Coupling cell growth, proliferation, and death. Hippo weighs in. Dev. Cell 5, 361-363.

Hay,B. A., Wassarman,D. A., and Rubin,G. M. (1995). Drosophila homologs of baculovirus inhibitor of apoptosis proteins function to block cell death. Cell 83, 1253-1262.

Hipffier,D. R. and Cohen,S. M. (2004). Connecting proliferation and apoptosis in development and disease. Nat. Rev. Mol. Cell Biol. 5, 805-815.

Hirota,T., Morisaki,T., Nishiyama,Y., Marumoto,T., Tada,K., Hara,T., Masuko,N., Inagaki,M., Hatakeyama,K., and Saya,H. (2000). Zyxin, a regulator of actin filament assembly, targets the mitotic apparatus by interacting with h-warts/LATS1 tumor suppressor. J. Cell Biol. 149, 1073-1086.

Hisaoka,M., Tanaka,A., and Hashimoto,H. (2002). Molecular alterations of h-warts/LATS1 tumor suppressor in human soft tissue sarcoma. Lab Invest 82, 1427-1435.

Jia,J., Zhang,W., Wang,B., Trinko,R., and Jiang,J. (2003). The Drosophila Ste20 family kinase dMST functions as a tumor suppressor by restricting cell proliferation and promoting apoptosis. Genes Dev. 17, 2514-2519.

Jones,L., Richardson,H., and Saint,R. (2000). Tissue-specific regulation of cyclin E transcription during Drosophila melanogaster embryogenesis. Development 127, 4619-4630.

Justice,R. W., Zilian,O., Woods,D. F., Noll,M., and Bryant,P. J. (1995). The Drosophila tumor suppressor gene warts encodes a homolog of human myotonic dystrophy kinase and is required for the control of cell shape and proliferation. Genes Dev. 9, 534-546.

Kango-Singh,M., Nolo,R., Tao,C., Verstreken,P., Hiesinger,P. R., Bellen,H. J., and Halder,G. (2002). Shar-pei mediates cell proliferation arrest during imaginal disc growth in Drosophila. Development 129, 5719-5730.

Lai,Z. C., Wei,X., Shimizu,T., Ramos,E., Rohrbaugh,M., Nikolaidis,N., Ho,L. L., and Li,Y. (2005). Control of Cell Proliferation and Apoptosis by Mob as Tumor Suppressor, Mats. Cell 120, 675-685.

Lowe,S. W., Cepero,E., and Evan,G. (2004). Intrinsic tumour suppression. Nature 432, 307-315.

Macias,M. J., Wiesner,S., and Sudol,M. (2002). WW and SH3 domains, two different scaffolds to recognize proline-rich ligands. FEBS Lett. 513, 30-37.

Mah,A. S., Jang,J., and Deshaies,R. J. (2001). Protein kinase Cdc15 activates the Dbf2-Mob1 kinase complex. Proc. Natl. Acad. Sci. U.S.A 98, 7325-7330.

Neufeld,T. P., de la Cruz,A. F. A., Johnston,L. A., and Edgar,B. A. (1998). Coordination of growth and cell division in the Drosophila wing. Cell 93, 1183-1193.

Newsome,T. P., Asling,B., and Dickson,B. J. (2000). Analysis of Drosophila photoreceptor axon guidance in eye-specific mosaics. Development 127, 851-860.

Pan,D., Dong,J., Zhang,Y., and Gao,X. (2004). Tuberous sclerosis complex: from Drosophila to human disease. Trends Cell Biol. 14, 78-85.

Pantalacci,S., Tapon,N., and Leopold,P. (2003). The Salvador partner Hippo promotes apoptosis and cell-cycle exit in Drosophila. Nat. Cell Biol. 5, 921-927.

Pignoni,F. and Zipursky,S. L. (1997). Induction of Drosophila eye development by decapentaplegic. Development 124, 271-278.

Rong,Y. S. and Golic,K. G. (2000). Gene targeting by homologous recombination in Drosophila. Science 288, 2013-2018.

Ryoo,H. D., Bergmann,A., Gonen,H., Ciechanover,A., and Steller,H. (2002). Regulation of Drosophila IAP1 degradation and apoptosis by reaper and ubcD1. Nat. Cell Biol. 4, 432-438.

Ryoo,H. D. and Steller,H. (2003). Hippo and its mission for growth control. Nat. Cell Biol. 5, 853-855.

St John,M. A., Tao,W., Fei,X., Fukumoto,R., Carcangiu,M. L., Brownstein,D. G., Parlow,A. F., McGrath,J., and Xu,T. (1999). Mice deficient of Latsl develop soft-tissue sarcomas, ovarian tumours and pituitary dysfunction. Nat. Genet. 21, 182-186.

Strano,S., Munarriz,E., Rossi,M., Castagnoli,L., Shaul,Y., Sacchi,A., Oren,M., Sudol,M., Cesareni,G., and Blandino,G. (2001). Physical interaction with Yes-associated protein enhances p73 transcriptional activity. J. Biol. Chem. 276, 15164-15173.

Struhl,G. and Basler,K. (1993). Organizing activity of wingless protein in Drosophila. Cell 72, 527-540.

Sudol,M. (1994). Yes-associated protein (YAP65) is a proline-rich phosphoprotein that binds to the SH3 domain of the Yes proto-oncogene product. Oncogene 9, 2145-2152.

Tamaskovic,R., Bichsel,S. J., and Hemmings,B. A. (2003). NDR family of AGC kinases—essential regulators of the cell cycle and morphogenesis. FEBS Lett. 546, 73-80.

Tao,W., Zhang,S., Turenchalk,G. S., Stewart,R. A., St John,M. A., Chen,W., and Xu,T. (1999). Human homologue of the Drosophila melanogaster lats tumour suppressor modulates CDC2 activity. Nat. Genet. 21, 177-181.

Tapon,N., Harvey,K. F., Bell,D. W., Wahrer,D. C., Schiripo,T. A., Haber,D. A., and Hariharan,I. K. (2002). salvador Promotes both cell cycle exit and apoptosis in Drosophila and is mutated in human cancer cell lines. Cell 110, 467-478.

Udan,R. S., Kango-Singh,M., Nolo,R., Tao,C., and Halder,G. (2003). Hippo promotes proliferation arrest and apoptosis in the Salvador/Warts pathway. Nat. Cell Biol. 5, 914-920.

Vassilev,A., Kaneko,K. J., Shu,H., Zhao,Y., and DePamphilis,M. L. (2001). TEAD/TEF transcription factors utilize the activation domain of YAP65, a Src/Yes-associated protein localized in the cytoplasm. Genes Dev. 15, 1229-1241.

Verde,F., Wiley,D. J., and Nurse,P. (1998). Fission yeast orb6, a ser/thr protein kinase related to mammalian rho kinase and myotonic dystrophy kinase, is required for maintenance of cell polarity and coordinates cell morphogenesis with the cell cycle. Proc. Natl. Acad. Sci. U.S.A 95, 7526-7531.

Wolff,T. and Ready,D. F. (1993). Pattern formation in the Drosophila retina. In The development of Drosophila melanogaster, M.Bate and A.Martinez Arias, eds. (Plainview, N.Y.: Cold Spring Harbor Laboratory Press), pp. 1277-1325.

Wu,S., Huang,J., Dong,J., and Pan,D. (2003). hippo encodes a Ste-20 family protein kinase that restricts cell proliferation and promotes apoptosis in conjunction with salvador and warts. Cell 114, 445-456.

Xu,T., Wang,W., Zhang,S., Stewart,R. A., and Yu,W. (1995). Identifying tumor suppressors in genetic mosaics: The Drosophila lats gene encodes a putative protein kinase. Development 121, 1053-1063.

Yagi,R., Chen,L. F., Shigesada,K., Murakami,Y., and Ito,Y. (1999). A WW domain-containing yes-associated protein (YAP) is a novel transcriptional co-activator. EMBO J. 18, 2551-2562.

Yang,X., Yu,K., Hao,Y., Li,D.M., Stewart,R., Insogna,K. L., and Xu,T. (2004). LATS1 tumour suppressor affects cytokinesis by inhibiting LIMK1. Nat. Cell Biol. 6, 609-617.

What is claimed is:

1. A method for diagnosing changes in cellular proliferation comprising the steps of:
determining the level of phosphorylation of a yes-associated protein (YAP) of a sample cell; and
comparing the level of phosphorylation of the yes-associated protein (YAP) in the sample cell to that of a normal cell, wherein a change in the level of phosphorylation is indicative of cell proliferation or cell death.

2. The method of claim 1, wherein the yes-associated protein (YAP) is phosphorylated by a Wts/Lats protein kinase.

3. The method of claim 1, wherein the YAP protein from the sample is used to measure transcription of cyclin E and diap1 in an in vitro translation reaction.

4. The method of claim 1, wherein the phosphorylation occurs in vitro.

5. The method of claim 1, wherein the yes-associated protein (YAP) comprises yorkie (ski).

6. The method of claim 1, wherein the yes-associated protein (YAP) comprises yorkie (yki) and yki overexpression leads to cell proliferation.

7. The method of claim 1, wherein the sample cell is obtained from a patient suspected of having cancer, an error in metabolism or a change in the phosphorylation state of the cell.

8. A method of identifying a polypeptide that interacts with a yes-associated protein (YAP), comprising the steps of:
contacting a polypeptide from a cDNA expression library with a Wts protein; and
identifying the polypeptide that has selective binding affinity for Wts.

9. The method of claim 8, wherein the yes-associated protein (YAP) is phosphorylated by a Wts/Lats protein kinase.

10. The method of claim 8, wherein the level of interaction between YAP and Wts is measured by the level of transcription of cyclin E, diap1 or both.

11. The method of claim 8, wherein the yes-associated protein (YAP) comprises yorkie (yki).

12. The method of claim 8, wherein determining the level of phosphorylation of the YAP indicates the state of signaling by an Hpo signaling pathway.

13. A kit for detecting the extent of interaction between a yes-associated protein (YAP) and a Wts protein comprising;
one or more vials comprising the YAP and the Wts protein; and
a kit for detecting the level of phosphorylation of the YAP protein that comprises one or more probes that detect phosphorylation.

14. A method for detecting an agent that interferes with the interaction between a yes-associated protein (YAP) and a Wts protein comprising;
contacting the YAP and the Wts proteins with the agent; and
detecting the level of phosphorylation of the YAP protein, wherein the level of phosphorylation is indicative of activation and the effect of the agent on the interaction between the YAP and the Wts proteins.

15. The method of claim 14, wherein YAP activity is detected functionally by in vitro translation of cyclin E, diap1 and combinations thereof.

16. A method for detecting oncogenesis comprising:
measuring the level of expression of a yorkie (yki) from a cell, wherein changes in the expression of YAP as compared to wild-type levels of the same cell-type is indicative of oncogenesis.

17. The method of claim 16, wherein YAP activity is detected functionally by in vitro translation of cyclin E, diap1 and combinations thereof.

18. An oncogene comprising an isolated and purified yki gene of SEQ ID NO.: 3 and complementary fragments thereof.

19. An isolated DNA segment that encodes an oncogenic polypeptide comprising the amino acid sequence of SEQ ID NO.: 2.

20. A vector comprising a nucleic acid that encodes an oncogene of SEQ ID NO.: 3.

21. A probe that binds specifically to SEQ ID NO.: 3 or the complement thereof.

22. A host cell comprising a nucleic acid of SEQ ID NO.: 3.

23. A method for modulating the size of an engineered organ comprising:
modifying the level of expression of a YAP protein.

24. The method of claim 23, wherein the nucleic acid of SEQ ID NO.: 3 is overexpressed to increase YAP activity to increase organ size.

25. The method of claim 23, wherein the nucleic acid of SEQ ID NO.: 3 is decreased to reduce YAP activity to decrease organ size.

26. A method for detecting oncogenicity comprising:
comparing the level of mRNA expression of a cell for SEQ ID NO.: 3 as compared to a wild-type cell, wherein an increase in the level of mRNA expression is indicative of oncogenesis.

27. The method of claim 26, wherein the oncogenic potential is confirmed by detecting a decrease in YAP phosphorylation, by decreased cellular apoptosis or both.


SEQUENCE LISTING (7 sequences)

SEQ ID NO: 1 - protein, 418 aa, Drosophila melanogaster
MCACLIAKIILCSFRLYTISAFYMLTTMSASSNTNSLIEKEIDDEDMLSPIKSNNLVVRVNQDTDDNLQALFDSVLNPGDAKRPLQLPLRMRKLPNSFFTPPAPSHSRANSADSTYDAGSQSSINIGNKASIVQQPDGQSPIAAIPQLQIQPSPQHSRLAIHHSRARSSPASLQQNYNVRARSDAAAANNPNANPSSQQQPAGPTFPENSAQEFPSGAPASSAIDLDAMNTCMSQDIPMSMQTVHKKQRSYDVISPIQLNRQLGALPPGWEQAKTNDGQIYYLNHTTKSTQWEDPRIQYRQQQQILMAERIKQNDVLQTTKQTTTSTIANNLGPLPDGWEQAVTESGDLYFINHIDRTTSWNDPRMQSGLSVLDCPDNLVSSLQIEDNLCSNLFNDAQAIVNPPSSHKPDDLEWYKIN

SEQ ID NO: 2 - protein, 504 aa, Homo sapiens
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 3 - DNA, 1539 bp, Homo sapiens
aagtggacggggatagccatctggcaacactgggataaatttattttatgttggcagttccgtaattattattattactattatttattgcaacgaagtttagtttttaacaccttaatgttatagtttcgcgcagcgcgatggagtgatacgtatatggaagaacatatatgtgcgcgtgcctaatcgctaagataattctatgtagttttcgtttgtatacaataagtgccttttatatgttaacgacgatgtcagccagcagcaatacaaacagcctgatcgagaaggagatcgacgacgaggacatgctttcgccgatcaagtccaacaacctggtggtgcgggtcaaccaggacacggacgacaacctgcaggcgctattcgacagcgtcctgaatccgggtgacgccaagcgcccgctgcagctgcccctgcgcatgcggaagctgcccaactccttcttcacgcccccggcgccctcgcactcgcgggccaacagcgccgactccacctacgacgcgggctcccagtcgagcatcaacatcgggaacaaggcgtccatcgtccagcagccagatggccagtcgcccatcgccgccatcccccagctccagattcagccgtctccccagcacagccgcctggcgatacatcactcccgagcccgcagcagccccgcctcgctgcagcagaactacaatgtgcgcgcccggagcgacgcagcagcagccaacaatccgaatgccaatccgagcagccaacagcagcccgctgggcccactttcccagagaacagtgcccaagagttccccagcggcgccccggccagctcggccattgatctggacgccatgaacacctgcatgtcgcaggacattcccatgtccatgcagacagtgcacaagaagcagcgctcctacgacgtcatcagccccattcagttgaaccgccaactaggcgccttgccgccgggatgggagcaagccaagaccaatgatggccagatctactacttgaatcatactacaaaatctacgcagtgggaggatcccagaatccaatatcgccagcagcagcaaatcttgatggccgagcgaataaagcagaatgatgttttgcaaactacaaaacaaactaccacatcgaccattgctaacaatttgggtccactgccggatggttgggagcaggcagttaccgagtccggagatctttactttataaatcacattgatcgaacgacttcatggaatgatcccagaatgcaatctgggcttagcgtgctcgactgcccagataacttagtgtcttccctccagattgaggataatctttgcagtaacttgttcaatgacgcacaggccattgtaaatccgccgtcttcccacaaacctgacgatttggaatggtataaaattaattaattcaatgtatacatctgtattagacctaaaagttttatattttgtattattctaaattaaatatttttcaaattttatagtatttctttacataaaaaaaaaaaaaaaaaaa

SEQ ID NO: 4 - DNA, 35 bp, Artificial sequence
  Synthetic oligonucleotide
agcaggcgcgccaatgtatacatctgtattagacc

SEQ ID NO: 5 - DNA, 33 bp, Artificial sequence
  Synthetic oligonucleotide
agcaggcgcgcccttacaaaacttttgccactg

SEQ ID NO: 6 - DNA, 34 bp, Artificial sequence
  Synthetic oligonucleotide
agcagcggccgcggggtgttagtagcttcaggtt

SEQ ID NO: 7 - DNA, 36 bp, Artificial sequence
  Synthetic oligonucleotide
agcagcggccgcatcttagcgattaggcacgcgcac